SEQUENCE LISTING (74 sequences)

SEQ ID NO: 1 - protein, 24 aa, Homo sapiens
  REGION 1..24: note = MBP epitope amino acids 83 to 106
DENPVVHFFKNIVTPRTPPPSQGK

SEQ ID NO: 2 - protein, 24 aa, Homo sapiens
  REGION 1..24: note = Candidate Antigen peptide
ADPGSRPHLIRLFSRDAPGREDNT

SEQ ID NO: 3 - protein, 10 aa, Homo sapiens
  REGION 1..10: note = Example Antigen Peptide
TQDENPVVHF

SEQ ID NO: 4 - protein, 10 aa, Homo sapiens
  REGION 1..10: note = Example Antigen Peptide
TQDQNPVVHF

SEQ ID NO: 5 - protein, 13 aa, Homo sapiens
  REGION 1..13: note = Example Epitope Peptide
GAEGQRPGFGYGG

SEQ ID NO: 6 - protein, 14 aa, Homo sapiens
  REGION 1..14: note = Example Database Entry Peptide
WGAEGQKPGFGYGG

SEQ ID NO: 7 - protein, 16 aa, Homo sapiens
  REGION 1..16: note = MBP epitope amino acids 87 to 102 of MBP isoform 5
VVHFFKNIVTPRTPPP

SEQ ID NO: 8 - protein, 22 aa, Homo sapiens
  REGION 1..22: note = MM1 epitope amino acids 270 to 291
LPQFEEIRNLALETLPAMCNVY

SEQ ID NO: 9 - protein, 10 aa, Homo sapiens
  REGION 1..10: note = MM1 epitope amino acids 279 to 288 with E282Q mutation
LALQTLPAMC

SEQ ID NO: 10 - protein, 171 aa, Homo sapiens
  REGION 1..171: note = Candidate Antigen
MASQKRPSQRHGSKYLATASTMDHARHGFLPRHRDTGILDSIGRFFGGDRGAPKRGSGKDSHHPARTAHYGSLPQKSHGRTQDENPVVHFFKNIVTPRTPPPSQGKGRGLSLSRFSWGAEGQRPGFGYGGRASDYKSAHKGFKGVDAQGTLSKIFKLGGRDSRSGSPMARR

SEQ ID NO: 11 - protein, 18 aa, Homo sapiens
  REGION 1..18: note = IGH CDR3
CAADGYSYGPRHNAFDIW

SEQ ID NO: 12 - protein, 18 aa, Homo sapiens
  REGION 1..18: note = IGH CDR3
CAAGTRSSGGSCYSLGYW

SEQ ID NO: 13 - protein, 17 aa, Homo sapiens
  REGION 1..17: note = IGH CDR3
CAAGYYYDSSGYDFQHW

SEQ ID NO: 14 - protein, 12 aa, Homo sapiens
  REGION 1..12: note = IGH CDR3
CAAIAAAGLAVW

SEQ ID NO: 15 - protein, 16 aa, Homo sapiens
  REGION 1..16: note = IGH CDR3
CAEDVGGYWVHQLGYW

SEQ ID NO: 16 - protein, 15 aa, Homo sapiens
  REGION 1..15: note = IGH CDR3
CAEEGGSGWPYFDYW

SEQ ID NO: 17 - protein, 17 aa, Homo sapiens
  REGION 1..17: note = IGH CDR3
CAEGRFGPYSSGWYASW

SEQ ID NO: 18 - protein, 15 aa, Homo sapiens
  REGION 1..15: note = IGH CDR3
CAGATVIPYNWFDPW

SEQ ID NO: 19 - protein, 17 aa, Homo sapiens
  REGION 1..17: note = IGH CDR3
CAGCPGGSSWYYYFDYW

SEQ ID NO: 20 - protein, 24 aa, Homo sapiens
  REGION 1..24: note = IGH CDR3
CAGDPPYCSNGVCSGPYYNGLDVW

SEQ ID NO: 21 - protein, 15 aa, Homo sapiens
  REGION 1..15: note = IGH CDR3
CAGELIAVAGPIDYW

SEQ ID NO: 22 - protein, 16 aa, Homo sapiens
  REGION 1..16: note = IGH CDR3
CAGRSSTAYYYIMDIW

SEQ ID NO: 23 - protein, 16 aa, Homo sapiens
  REGION 1..16: note = IGH CDR3
CAGRSSTAYYYTMDIW

SEQ ID NO: 24 - protein, 21 aa, Homo sapiens
  REGION 1..21: note = IGH CDR3
CAGVSYYYDSSGYYYEPFDYW

SEQ ID NO: 25 - protein, 13 aa, Homo sapiens
  REGION 1..13: note = IGH CDR3
CAHGKLAGPFDSW

SEQ ID NO: 26 - protein, 14 aa, Homo sapiens
  REGION 1..14: note = IGH CDR3
CAHGRYLDGAIDYW

SEQ ID NO: 27 - protein, 14 aa, Homo sapiens
  REGION 1..14: note = IGH CDR3
CAHKKLFGELPDYW

SEQ ID NO: 28 - protein, 20 aa, Homo sapiens
  REGION 1..20: note = IGH CDR3
CAHLTITFGGTPRDDAFDSW

SEQ ID NO: 29 - protein, 18 aa, Homo sapiens
  REGION 1..18: note = IGH CDR3
CAHRLGPLANRAAYFDYW

SEQ ID NO: 30 - protein, 15 aa, Homo sapiens
  REGION 1..15: note = IGH CDR3
CAHRQGYSYGIADYW

SEQ ID NO: 31 - protein, 18 aa, Homo sapiens
  REGION 1..18: note = MBP Amino acid alignment from Adaptive Match
FLPRHRDTGILDSIGRFF

SEQ ID NO: 32 - protein, 18 aa, Homo sapiens
  REGION 1..18: note = MBP Amino acid alignment from Adaptive Match
GFKGVDAQGTLSKIFKLG

SEQ ID NO: 33 - protein, 17 aa, Homo sapiens
  REGION 1..17: note = MBP Amino acid alignment from Adaptive Match
PVVHFFKNIVTPRTPPP

SEQ ID NO: 34 - protein, 12 aa, Homo sapiens
  REGION 1..12: note = MBP Amino acid alignment from Adaptive Match
PVVHFFKNIVTP

SEQ ID NO: 35 - protein, 16 aa, Homo sapiens
  REGION 1..16: note = MBP Amino acid alignment from Adaptive Match
LPRHRDTGILDSIGRF

SEQ ID NO: 36 - protein, 15 aa, Homo sapiens
  REGION 1..15: note = MBP Amino acid alignment from Adaptive Match
GFKGVDAQGTLSKIF

SEQ ID NO: 37 - protein, 17 aa, Homo sapiens
  REGION 1..17: note = MBP Amino acid alignment from Adaptive Match
FKGVDAQGTLSKIFKLG

SEQ ID NO: 38 - protein, 15 aa, Homo sapiens
  REGION 1..15: note = MBP Amino acid alignment from Adaptive Match
GVDAQGTLSKIFKLG

SEQ ID NO: 39 - protein, 17 aa, Homo sapiens
  REGION 1..17: note = MBP Amino acid alignment from Adaptive Match
FKGVDAQGTLSKIFKLG

SEQ ID NO: 40 - protein, 24 aa, Homo sapiens
  REGION 1..24: note = MBP Amino acid alignment from Adaptive Match
YKSAHKGFKGVDAQGTLSKIFKLG

SEQ ID NO: 41 - protein, 15 aa, Homo sapiens
  REGION 1..15: note = MBP Amino acid alignment from Adaptive Match
GFKGVDAQGTLSKIF

SEQ ID NO: 42 - protein, 16 aa, Homo sapiens
  REGION 1..16: note = MBP Amino acid alignment from Adaptive Match
KGVDAQGTLSKIFKLG

SEQ ID NO: 43 - protein, 21 aa, Homo sapiens
  REGION 1..21: note = MBP Amino acid alignment from Adaptive Match
TGILDSIGRFFGGDRGAPKRG

SEQ ID NO: 44 - protein, 13 aa, Homo sapiens
  REGION 1..13: note = MBP Amino acid alignment from Adaptive Match
FKGVDAQGTLSKI

SEQ ID NO: 45 - protein, 14 aa, Homo sapiens
  REGION 1..14: note = MBP Amino acid alignment from Adaptive Match
VDAQGTLSKIFKLG

SEQ ID NO: 46 - protein, 20 aa, Homo sapiens
  REGION 1..20: note = MBP Amino acid alignment from Adaptive Match
GILDSIGRFFGGDRGAPKRG

SEQ ID NO: 47 - protein, 18 aa, Homo sapiens
  REGION 1..18: note = MBP Amino acid alignment from Adaptive Match
ILDSIGRFFGGDRGAPKR

SEQ ID NO: 48 - protein, 15 aa, Homo sapiens
  REGION 1..15: note = MBP Amino acid alignment from Adaptive Match
GVDAQGTLSKIFKLG

SEQ ID NO: 49 - protein, 4 aa, Homo sapiens
  REGION 1..4: note = Proteolipid Protein Antigen
LYGA

SEQ ID NO: 50 - protein, 14 aa, Homo sapiens
  REGION 1..14: note = WURR-Combo CS for MBP isoform 5 range 118-131
GAEGQRPGFGYGGR

SEQ ID NO: 51 - protein, 16 aa, Homo sapiens
  REGION 1..16: note = IGH CDR3
CARVLDWRAGSPTSPW

SEQ ID NO: 52 - protein, 27 aa, Homo sapiens
  REGION 1..27: note = IGH CDR3
CARRMTVVAEYNFWSSYSSGPSWFDPW

SEQ ID NO: 53 - protein, 27 aa, Homo sapiens
  REGION 1..27: note = IGH CDR3 Compliment Sequence
QDENPVVHFFKNIVTPRTPPPSQGKGR

SEQ ID NO: 54 - protein, 16 aa, Homo sapiens
  REGION 1..16: note = IGH CDR3
CAKTRPHLVLVTVPVW

SEQ ID NO: 55 - protein, 16 aa, Homo sapiens
  REGION 1..16: note = IGH CDR3 Compliment Sequence
TQDENPVVHFFKNIVT

SEQ ID NO: 56 - protein, 27 aa, Homo sapiens
  REGION 1..27: note = Alpha/Beta Gliadin MM1 antigen peptide
TFLILALLAIVATTARIAVRVPVPQLQ

SEQ ID NO: 57 - protein, 17 aa, Homo sapiens
  REGION 1..17: note = Alpha/Beta Gliadin MM1 antigen peptide
IPCRDVVLQQHSIAYGS

SEQ ID NO: 58 - protein, 18 aa, Homo sapiens
  REGION 1..18: note = Alpha/Beta Gliadin MM1 antigen peptide
IPEQSRCQAIHNVVHAII

SEQ ID NO: 59 - protein, 29 aa, Homo sapiens
  REGION 1..29: note = Prolamin antigen peptide
QILQQILQQQLIPCRDVVLQQPNIAHASS

SEQ ID NO: 60 - protein, 13 aa, Homo sapiens
  REGION 1..13: note = Prolamin antigen peptide
IHNVIHAIILHHQ

SEQ ID NO: 61 - protein, 50 aa, Homo sapiens
  REGION 1..50: note = Prolamin antigen peptide
NPQAQGFVQPQQLPQFEEIRNLALQTLPAMCNVYIPPYCSTTIAPFGIFS

SEQ ID NO: 62 - protein, 22 aa, Homo sapiens
  REGION 1..22: note = Gamma Gliadin antigen peptide
TLLILTILAMAITIGTANIQVD

SEQ ID NO: 63 - protein, 21 aa, Homo sapiens
  REGION 1..21: note = Gamma Gliadin antigen peptide
QGIDIFLPLSQHEQVGQGSLV

SEQ ID NO: 64 - protein, 51 aa, Homo sapiens
  REGION 1..51: note = Gamma Gliadin antigen peptide
GIIQPQQPAQLEAIRSLVLQTLPSMCNVYVPPECSIMRAPFASIVAGIGGQ

SEQ ID NO: 65 - protein, 21 aa, Homo sapiens
  REGION 1..21: note = Gamma Gliadin antigen peptide
HRDGVRRPQKRPSCIGCKGTH

SEQ ID NO: 66 - protein, 5 aa, Homo sapiens
  REGION 1..5: note = Gamma Gliadin antigen peptide
YNLRR

SEQ ID NO: 67 - protein, 23 aa, Homo sapiens
  REGION 1..23: note = Gamma Gliadin antigen peptide
DGVDLPPWFPPMVEGAAAEGDDG

SEQ ID NO: 68 - protein, 18 aa, Homo sapiens
  REGION 1..18: note = Gamma Gliadin antigen peptide
GGDNHGRGRGRGRGRGGG

SEQ ID NO: 69 - protein, 9 aa, Homo sapiens
  REGION 1..9: note = Gamma Gliadin antigen peptide
EGTWVAGVF

SEQ ID NO: 70 - protein, 24 aa, Homo sapiens
  REGION 1..24: note = Gamma Gliadin antigen peptide
YNLRRGTALAIPQCRLTPLSRLPF

SEQ ID NO: 71 - protein, 11 aa, Homo sapiens
  REGION 1..11: note = Gamma Gliadin antigen peptide
GPLRESIVCYF

SEQ ID NO: 72 - protein, 6 aa, Homo sapiens
  REGION 1..6: note = Gamma Gliadin antigen peptide
VFLQTH

SEQ ID NO: 73 - protein, 20 aa, Homo sapiens
  REGION 1..20: note = Gamma Gliadin antigen peptide
KDAIKDLVMTKPAPTCNIRV

SEQ ID NO: 74 - protein, 15 aa, Homo sapiens
  REGION 1..15: note = Gamma Gliadin antigen peptide
FPPMVEGAAAEGDDG

(19) United States
(12) Patent Application Publication
(10) Pub. No.: US 2025/0279160 A1
(43) Pub. Date: Sep. 4, 2025

(54) Methods of Using Chemical Complementarity Scoring

(71) Applicant: University of South Florida, Tampa, FL (US)

(72) Inventors: Justin Cole, Tampa, FL (US); George Blanck, Tampa, FL (US)

(21) Appl. No.: 19/066,713

(22) Filed: Feb. 28, 2025

Related U.S. Application Data
(60) Provisional application No. 63/563,571, filed on Mar. 11, 2024.
(60) Provisional application No. 63/559,911, filed on Mar. 1, 2024.

(51) Int. Cl. G16B 15/30; G01N 33/564; G01N 33/68; G16B 45/00; G16H 15/00; G16H 50/20

(52) U.S. Cl. CPC G16B 15/30; G01N 33/564; G01N 33/6857; G16B 45/00; G16H 15/00; G16H 50/20; G01N 2800/24; G01N 2800/285; G01N 2800/52

(57) ABSTRACT

The present disclosure relates methods of treating, preventing, and/or diagnosing autoimmune diseases using chemical complementarity scoring.

### CROSS-REFERENCE TO RELATED APPLICATION

This US Utility application claims priority to, and the benefit of, U.S. Provisional Patent Application No. 63/559,911, filed Mar. 1, 2024, entitled “CHEMICAL COMPLEMENTARITY SCORING AS A COMPUTATIONAL APPROACH TO MATCHING MULTIPLE SCLEROSIS RELATED IGH CDR3s WITH A MYELIN BASIC PROTEIN EPITOPE,” and U.S. Provisional Patent Application No. 63/563,571, filed Mar. 11, 2024, entitled “CHEMICAL COMPLEMENTARITY SCORING AS A COMPUTATIONAL APPROACH TO MATCHING MULTIPLE SCLEROSIS RELATED IGH CDR3s WITH A MYELIN BASIC PROTEIN EPITOPE,” which is incorporated by reference herein in its entirety.

### REFERENCE TO SEQUENCE LISTING

The sequence listing submitted on Mar. 1, 2025, as an .XML file entitled “11001-211US1-ST26” created on Feb. 20, 2025, and having a file size of 94,174 bytes is hereby incorporated by reference pursuant to 37 C.F.R. § 1.52(e)(5).

### FIELD

The present disclosure relates methods of treating, preventing, and/or diagnosing autoimmune diseases using chemical complementarity scoring.

### BACKGROUND

Autoimmune diseases are a diverse group of conditions characterized by aberrant T cell and/or B cell reactivity to a subject's tissues and cells. These diseases occur widely and affect individuals of all ages and ethnicities. Among these diseases, the most prominent immunological manifestation is the production of autoantibodies, which could provide valuable biomarkers for disease diagnosis, classification, and disease activity. Current treatments for autoimmune disease include targeted immunotherapies that lead to suppression of major pro-inflammatory signaling pathways by blocking inflammatory cytokines, cell surface molecules, and intracellular kinases. Despite these recent advancements in treatment, there remains an unmet need to successfully distinguish patients suffering from an autoimmune disease from other individuals not suffering for an autoimmune disease. Furthermore, efficient computational analyses to diagnose or monitor autoimmune diseases, which could have broad applicability in clinical trials or in diagnoses, remains a challenge.

Given the limitations described above, there remains a need to develop an efficient method of preventing, diagnosing, treating, and/or monitoring autoimmune diseases. The present disclosure addresses these needs and more.

### SUMMARY

The present disclosure provides treating, preventing, and/or diagnosing autoimmune diseases, including, but not limited to multiple sclerosis and celiac disease, using chemical complementarity scoring.

In some aspects, disclosed herein is a method of treating or preventing an autoimmune disease in a subject, the method comprising collecting a sample from the subject, identifying one or more immunoglobulin heavy chain (IGH) complementarity determining regions (CDR) 3 within the sample, determining a complementarity score (CS) between the IGH CDR3 and an epitope of the autoimmune disease, wherein the CS is based on electrostatic and hydrophobic interactions between the IGH CDR3 and the epitope and administering a therapeutic agent to the subject when the CS score is increased relative to a control subject.

In some aspects, disclosed herein is a method of diagnosing a subject with an autoimmune disease in a subject, the method comprising collecting a sample from the subject, identifying one or more immunoglobulin heavy chain (IGH) complementarity determining regions (CDR) 3 within the sample, determining a complementarity score (CS) between the IGH CDR3 and an epitope of the autoimmune disease, wherein the CS is based on electrostatic and hydrophobic interactions between the IGH CDR3 and the epitope, and diagnosing the subject with the autoimmune disease when the CS score is increased relative to a control subject.

In some embodiments, the autoimmune disease comprises Multiple Sclerosis (MS) or celiac disease. In some embodiments, the subject is administered the therapeutic agent when the CS score is 6.0 or more. In some embodiments, the epitope comprises a whole antigen peptide. In some embodiments, the epitope comprises a partial antigen peptide. In some embodiments, the therapeutic agent comprises an immunotherapeutic agent, a muscle relaxant agent, an analgesic, a plasma composition, a cell-based composition, or a combination thereof. In some embodiments, the sample is a blood sample.

In some aspects, disclosed herein is a computer-implemented method comprising obtaining or determining, by at least one processor, an immune repertoire for a subject's blood sample, programmatically identifying, by the at least one processor, one or more candidate epitopes corresponding with at least one known or unknown autoimmune disease, by using at least one chemical complementarity algorithm to determine a ratio or value indicating a number of times each of the one or more candidate epitopes complements one of a plurality of amino acids, and determining, by the at least one processor, a disease state or condition of the subject and/or isolating at least one target epitope based, at least in part, on a frequency count and/or degree of correspondence between each respective candidate epitope and respective amino acid.

In some embodiments, the computer-implemented method comprises isolating at least one target epitope and further determining a statistical significance of the at least one target epitope based, at least in part, on a difference in weighted unique residue ratio (WURR) values outside the at least one target epitope relative to one or more control samples.

In some embodiments, identifying the one or more candidate epitopes comprises applying a sliding window analysis with respect to the one or more candidate epitopes and the plurality of amino acids. In some embodiments, the computer-implemented method further comprises generating user interface data (e.g., graphical information, a report) based on the determined disease state or condition of the subject and/or isolated target epitope.

In some aspects, disclosed herein is a system comprising at least one processor and a memory operably coupled to the at least one processor, wherein the memory has computer executable instructions stored thereon that, when executed by the at least one processor, cause the at least one processor to obtain or determine an immune repertoire for a subject's blood sample, programmatically identify one or more candidate epitopes corresponding with at least one known or unknown autoimmune disease, by using at least one chemical complementarity algorithm to determine a ratio or value indicating a number of times each of the one or more candidate epitopes complements one of a plurality of amino acids, determine a disease state or condition of the subject and/or isolating at least one target epitope based, at least in part, on a frequency count and/or degree of correspondence between each respective candidate epitope and respective amino acid.

### BRIEF DESCRIPTION OF FIGURES

The accompanying figures, which are incorporated in and constitute a part of this specification, illustrate several aspects described below.

FIG. 1 shows the average frequency count of highly, chemically complementary CDR3s, with respect to the canonical MBP epitope. The average number of CDR3s with a Combo CS of 6.0 or greater overlapping each MBP residue of the canonical epitope for MS samples (solid line, n=8); the average number of CDR3s with Combo CS of 6.0 or greater overlapping each MBP residue of the canonical epitope for control samples (dashed line, n=8). These data showed that the statistically significant results seen in the Mann-Whitney (Table 7) and Chi-squared (Table 9) analyses are primarily traceable to the IGH CDR3-MBP canonical epitope pairs overlapping the valine at position 87 to the valine at position 95.

FIG. 2 shows the average frequency count of highly, chemically complementary CDR3s, with respect to the novel candidate MBP epitope. The average number of CDR3s with Combo CS of 6.0 or greater overlapping each MBP residue of the candidate epitope for MS samples (solid line, n=8); the average number of CDR3s with Combo CS of 6.0 or greater overlapping each MBP residue of the candidate epitope for control samples (dashed line, n=8). These data showed that the statistically significant results seen previously in the Mann-Whitney (Table 11) and Chi-squared (Table 12) analyses are primarily traceable to the IGH CDR3-MBP, novel candidate epitope pairs overlapping the alanine at position 83 to the serine at position 96.

FIGS. 3A, 3B, and 3C show the de novo epitope flowcharts and an operational example. FIG. 3A shows the flowchart depicting the algorithmic process outlined in this study that leads to defining candidate epitopes. FIG. 3B shows an example computer-implemented method in accordance with certain embodiments disclosed herein. FIG. 3C is an operational example of a user interface.

FIGS. 4A and 4B show the Weighted Unique Residue Ratios (WURR) of MS, Healthy, and COVID samples over the length of MBP isoform 5. FIG. 4A shows the WURR value of each residue for MS samples over the length of the MBP isoform 5 antigen (black shaded region, n=8); the WURR value of each residue for COVID samples over the length of the MBP isoform 5 antigen (gray shaded region, n=8). These data showed that there was an elevated difference in WURR values over the indicated regions (Table 20). FIG. 4B shows the WURR value of each residue for MS samples over the length of the MBP isoform 5 antigen (black shaded region, n=8); the WURR value of each residue for Healthy samples over the length of the MBP isoform 5 antigen (gray shaded region, n=8). These data showed that there was an elevated difference in WURR values over the indicated regions (Table 20).

FIG. 5 shows the Weighted Unique Residue Ratios (WURR) of CD and Healthy samples over the length of MBP isoform 5. The WURR value of each residue for CD samples over the length of the MBP isoform 5 antigen (black shaded region, n=8); the WURR value of each residue for Healthy samples over the length of the MBP isoform 5 antigen (gray shaded region, n=8). These data showed that there were no elevated differences in WURR values over the length of the antigen.

FIG. 6 shows the Weighted Unique Residue Ratios (WURR) of CD and Healthy samples over the length of alpha/beta-gliadin MM1. The WURR value of each residue for CD samples over the length of the alpha/beta-gliadin MM1 antigen (black shaded region, n=8); the WURR value of each residue for Healthy samples over the length of the alpha/beta-gliadin MM1 antigen (gray shaded region, n=8). These data showed that there was an elevated difference in WURR values over the indicated regions (Tables 23 and 24).

FIGS. 7A and 7B show the Weighted Unique Residue Ratios (WURR) of MS, Healthy and COVID samples over the length of EBV nuclear antigen 1. The WURR value of each residue for MS samples over the length of the EBV nuclear antigen 1 (black shaded region, n=8); the WURR value of each residue for COVID samples over the length of the EBV nuclear antigen 1 (gray shaded region, n=8); These data showed that there was an elevated difference in WURR values over the indicated regions (Tables 23 and 24). The WURR value of each residue for MS samples over the length of the EBV nuclear antigen 1 (black shaded region, n=8); the WURR value of each residue for Healthy samples over the length of the EBV nuclear antigen 1 (gray shaded region, n=8); These data showed that there was an elevated difference in WURR values over the indicated regions (Tables 23 and 24).

FIG. 8 shows an example computing device.

### DETAILED DESCRIPTION

The following description of the disclosure is provided as an enabling teaching of the disclosure in its best, currently known embodiment(s). To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

Reference will now be made in detail to the embodiments of the invention, examples of which are illustrated in the drawings and the examples. This invention may, however, be embodied in many different forms and should not be construed as limited to the embodiments set forth herein.

### Terminology

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a”, “an”, “the”, include plural referents unless the context clearly dictates otherwise.

The following definitions are provided for the full understanding of terms used in this specification.

The terms “about” and “approximately” are defined as being “close to” as understood by one of ordinary skill in the art. In one non-limiting embodiment the terms are defined to be within 10%. In another non-limiting embodiment, the terms are defined to be within 5%. In still another non-limiting embodiment, the terms are defined to be within 1%.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., an autoimmune disease). The terms also encompass pharmaceutically acceptable, pharmacologically active derivatives of beneficial agents specifically mentioned herein, including, but not limited to, a vector, polynucleotide, cells, salts, esters, amides, proagents, active metabolites, isomers, fragments, analogs, and the like. When the term “composition” is used, then, or when a particular composition is specifically identified, it is to be understood that the term includes the composition per se as well as pharmaceutically acceptable, pharmacologically active vector, polynucleotide, salts, esters, amides, proagents, conjugates, active metabolites, isomers, fragments, analogs, etc.

An “increase” can refer to any change that results in a greater amount of a symptom, disease, composition, condition, or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, 100% or more increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, 100%, or more decrease so long as the decrease is statistically significant.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The terms “treat,” “treating,” and grammatical variations thereof as used herein, include partially or completely delaying, alleviating, mitigating or reducing the intensity of one or more attendant symptoms of a disorder or condition and/or alleviating, mitigating or impeding one or more causes of a disorder or condition. Treatments according to the disclosure may be applied preventively, prophylactically, palliatively or remedially. Treatments are administered to a subject prior to onset (e.g., before obvious signs of inflammation, pain, and/or other symptoms associated with autoimmune diseases), during early onset (e.g., upon initial signs and symptoms of inflammation, pain, and/or other symptoms associated with autoimmune diseases), or after an established development of inflammation, pain, and/or other symptoms associated with autoimmune diseases.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

A “patient” is any subject receiving or awaiting to receive medical care or treatment. A “patient” can be a human, non-human primate, non-human mammal, or any other vertebrate or non-vertebrate animal. For example, a patient can be a human, a dog, a cat, a monkey, an ape, a bird, a frog, a mouse, a rabbit, a fish, a jellyfish, or snake.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

The term “amino acid,” includes but is not limited to amino acids contained in the group consisting of alanine (Ala or A), cysteine (Cys or C), aspartic acid (Asp or D), glutamic acid (Glu or E), phenylalanine (Phe or F), glycine (Gly or G), histidine (His or H), isoleucine (Ile or I), lysine (Lys or K), leucine (Leu or L), methionine (Met or M), asparagine (Asn or N), proline (Pro or P), glutamine (Gln or Q), arginine (Arg or R), serine (Ser or S), threonine (Thr or T), valine (Val or V), tryptophan (Trp or W), and tyrosine (Tyr or Y) residues. The term “amino acid residue” also may include amino acid residues contained in the group consisting of homocysteine, 2-Aminoadipic acid, N-Ethylasparagine, 3-Aminoadipic acid, Hydroxylysine, β-alanine, β-Amino-propionic acid, allo-Hydroxylysine acid, 2-Aminobutyric acid, 3-Hydroxyproline, 4-Aminobutyric acid, 4-Hydroxyproline, piperidinic acid, 6-Aminocaproic acid, Isodesmosine, 2-Aminoheptanoic acid, allo-Isoleucine, 2-Aminoisobutyric acid, N-Methylglycine, sarcosine, 3-Aminoisobutyric acid, N-Methylisoleucine, 2-Aminopimelic acid, 6-N-Methyllysine, 2,4-Diaminobutyric acid, N-Methylvaline, Desmosine, Norvaline, 2,2′-Diaminopimelic acid, Norleucine, 2,3-Diaminopropionic acid, Ornithine, and N-Ethylglycine. Typically, the amide linkages of the peptides are formed from an amino group of the backbone of one amino acid and a carboxyl group of the backbone of another amino acid.

Reference also is made herein to peptides, polypeptides, proteins, and compositions comprising peptides, polypeptides, and proteins. As used herein, a polypeptide and/or protein is defined as a polymer of amino acids, typically of length≥100 amino acids (Garrett & Grisham, Biochemistry, 2nd edition, 1999, Brooks/Cole, 110). A peptide is defined as a short polymer of amino acids, of a length typically of 20 or less amino acids, and more typically of a length of 12 or less amino acids (Garrett & Grisham, Biochemistry, 2nd edition, 1999, Brooks/Cole, 110).

The peptides, polypeptides, and proteins disclosed herein may be modified to include non-amino acid moieties. Modifications may include but are not limited to carboxylation (e.g., N-terminal carboxylation via addition of a di-carboxylic acid having 4-7 straight-chain or branched carbon atoms, such as glutaric acid, succinic acid, adipic acid, and 4,4-dimethylglutaric acid), amidation (e.g., C-terminal amidation via addition of an amide or substituted amide such as alkylamide or dialkylamide), PEGylation (e.g., N-terminal or C-terminal PEGylation via additional of polyethylene glycol), acylation (e.g., O-acylation (esters), N-acylation (amides), S-acylation (thioesters)), acetylation (e.g., the addition of an acetyl group, cither at the N-terminus of the protein or at lysine residues), formylation lipoylation (e.g., attachment of a lipoate, a C8 functional group), myristoylation (e.g., attachment of myristate, a C14 saturated acid), palmitoylation (e.g., attachment of palmitate, a C16 saturated acid), alkylation (e.g., the addition of an alkyl group, such as an methyl at a lysine or arginine residue), isoprenylation or prenylation (e.g., the addition of an isoprenoid group such as farnesol or geranylgeraniol), amidation at C-terminus, glycosylation (e.g., the addition of a glycosyl group to either asparagine, hydroxylysine, serine, or threonine, resulting in a glycoprotein). Distinct from glycation, which is regarded as a nonenzymatic attachment of sugars, polysialylation (e.g., the addition of polysialic acid), glypiation (e.g., glycosylphosphatidylinositol (GPI) anchor formation, hydroxylation, iodination (e.g., of thyroid hormones), and phosphorylation (e.g., the addition of a phosphate group, usually to serine, tyrosine, threonine, or histidine).

The phrases “percent identity” and “% identity,” as applied to polypeptide sequences, refer to the percentage of residue matches between at least two polypeptide sequences aligned using a standardized algorithm. Methods of polypeptide sequence alignment are well-known. Some alignment methods consider conservative amino acid substitutions. Such conservative substitutions, explained in more detail above, generally preserve the charge and hydrophobicity at the site of substitution, thus preserving the structure (and therefore function) of the polypeptide. Percent identity for amino acid sequences may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403 410), which is available from several sources, including the NCBI, Bethesda, Md., at its website. The BLAST software suite includes various sequence analysis programs including “blastp,” that is used to align a known amino acid sequence with other amino acids sequences from a variety of databases. Percent identity may be measured over the length of an entire defined polypeptide sequence or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length may be used to describe a length over which percentage identity may be measured.

The term “variant” means a polypeptide derived from a parent polypeptide by one or more (several) alteration(s), i.e., a substitution, insertion, and/or deletion, at one or more (several) positions. A substitution means a replacement of an amino acid occupying a position with a different amino acid; a deletion means removal of an amino acid occupying a position; and an insertion means adding I or more, such as 1, 2, 3, 4, 5, 6, 7, 8, 9 or 10, preferably 1-3 amino acids immediately adjacent an amino acid occupying a position. In relation to substitutions, ‘immediately adjacent’ may be to the N-side (‘upstream’) or C-side (‘downstream’) of the amino acid occupying a position (‘the named amino acid’). Therefore, for an amino acid named/numbered ‘X,’ the insertion may be at position ‘X+1’ (‘downstream’) or at position ‘X−1’ (‘upstream’).

A “variant” of a particular polypeptide sequence may be defined as a polypeptide sequence having at least 50% sequence identity to the particular polypeptide sequence over a certain length of one of the polypeptide sequences using blastp with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). In some embodiments a variant polypeptide may show, for example, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length relative to a reference polypeptide. A variant polypeptide may have substantially the same functional activity as a reference polypeptide. For example, a variant polypeptide may exhibit or more biological activities associated with binding a ligand and/or binding DNA at a specific binding site.

The term “administer,” “administering”, or derivatives thereof refer to delivering a composition, substance, inhibitor, or medication to a subject or object by one or more the following routes: oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation or via an implanted reservoir. The term “parenteral” includes subcutaneous, intravenous, intramuscular, intra-articular, intra-synovial, intrasternal, intrathecal, intrahepatic, intralesional, and intracranial injections or infusion techniques.

The term “detect” or “detecting” refers to an output signal released for the purpose of sensing of physical phenomenon. For example, an event or change in environment is sensed and signal output released in the form of light.

The term “antibody” is used in the broadest sense, and specifically covers monoclonal antibodies (including full length monoclonal antibodies), polyclonal antibodies, and multispecific antibodies (e.g., bispecific antibodies). Antibodies (Abs) and immunoglobulins (Igs) are glycoproteins having the same structural characteristics. While antibodies exhibit binding specificity to a specific target, immunoglobulins include both antibodies and other antibody-like molecules which lack target specificity. Native antibodies and immunoglobulins are usually heterotetrameric glycoproteins of about 150,000 daltons, composed of two identical light (L) chains and two identical heavy (H) chains. Each heavy chain has at one end a variable domain (V_{H}) followed by a number of constant domains. Each light chain has a variable domain at one end (V_{L}) and a constant domain at its other end.

The term “antibody fragment” refers to a portion of a full-length antibody, generally the target binding or variable region. Examples of antibody fragments include Fab, Fab′, F(ab′)₂ and Fv fragments. The phrase “functional fragment or analog” of an antibody is a compound having qualitative biological activity in common with a full-length antibody. For example, a functional fragment or analog of an anti-IgE antibody is one which can bind to an IgE immunoglobulin in such a manner so as to prevent or substantially reduce the ability of such molecule from having the ability to bind to the high affinity receptor, FcεRI. As used herein, “functional fragment” with respect to antibodies, refers to Fv, F (ab) and F(ab′)₂ fragments. An “Fv” fragment is the minimum antibody fragment which contains a complete target recognition and binding site. This region consists of a dimer of one heavy and one light chain variable domain in a tight, non-covalent association (V_{H}-V_{L} dimer). It is in this configuration that the three CDRs of each variable domain interact to define a target binding site on the surface of the V_{H}-V_{L} dimer. Collectively, the six CDRs confer target binding specificity to the antibody. However, even a single variable domain (or half of an Fv comprising only three CDRs specific for a target) has the ability to recognize and bind target, although at a lower affinity than the entire binding site. “Single-chain Fv” or “sFv” antibody fragments comprise the V_{H} and V_{L} domains of an antibody, wherein these domains are present in a single polypeptide chain. Generally, the Fv polypeptide further comprises a polypeptide linker between the V_{H} and V_{L} domains which enables the sFv to form the desired structure for target binding.

The terms “immunotherapy” and “immunotherapeutic” refers to the treatment of disease by activating or suppressing the immune system. In cancer treatment, the most effective immunotherapies are cell-based immunotherapies that utilize lymphocytes, macrophages, dendritic cells, natural killer cells, cytotoxic T lymphocytes, etc. to defend the body against cancer by targeting abnormal antigens expressed on the surface of tumor cells.

The term “variable” in the context of variable domain of antibodies, refers to the fact that certain portions of the variable domains differ extensively in sequence among antibodies and are used in the binding and specificity of each particular antibody for its particular target. However, the variability is not evenly distributed through the variable domains of antibodies. It is concentrated in three segments called complementarity determining regions (CDRs) also known as hypervariable regions both in the light chain and the heavy chain variable domains. The more highly conserved portions of variable domains are called the framework (FR). The variable domains of native heavy and light chains each comprise four FR regions, largely a adopting a .beta.-sheet configuration, connected by three CDRs, which form loops connecting, and in some cases forming part of, the .beta.-sheet structure. The CDRs in each chain are held together in close proximity by the FR regions and, with the CDRs from the other chain, contribute to the formation of the target binding site of antibodies (see Kabat et al.) As used herein, numbering of immunoglobulin amino acid residues is done according to the immunoglobulin amino acid residue numbering system of Kabat et al., (Sequences of Proteins of Immunological Interest, National Institute of Health, Bethesda, Md. 1987), unless otherwise indicated.

An “epitope” or “antigenic determinant” refer to the part of an antigen, a molecular structure, or foreign particulate that can bind to a specific antibody or T-cell receptor. The presence of antigens or epitopes of antigens within a host can illicit an immune response.

An “antigen” refers to a molecule, moiety, foreign particulate matter, or an allergen that can bind to a specific antibody or T cell receptor. The presence of antigens within a host can illicit an immune response against said molecule, moiety, foreign particulate matter, or allergen.

### Methods of Using Chemical Complementarity Scoring.

The present disclosure provides treating, preventing, and/or diagnosing autoimmune diseases, including, but not limited to multiple sclerosis and celiac disease, using chemical complementarity scoring.

In some aspects, disclosed herein is a method of treating or preventing an autoimmune disease in a subject, the method comprising collecting a sample from the subject, identifying one or more immunoglobulin heavy chain (IGH) complementarity determining regions (CDR) 3 within the sample, determining a complementarity score (CS) between the IGH CDR3 and an epitope of the autoimmune disease, wherein the CS is based on electrostatic and hydrophobic interactions between the IGH CDR3 and the epitope and administering a therapeutic agent to the subject when the CS score is increased relative to a control subject.

In some aspects, disclosed herein is a method of diagnosing a subject with an autoimmune disease in a subject, the method comprising collecting a sample from the subject, identifying one or more immunoglobulin heavy chain (IGH) complementarity determining regions (CDR) 3 within the sample, determining a complementarity score (CS) between the IGH CDR3 and an epitope of the autoimmune disease, wherein the CS is based on electrostatic and hydrophobic interactions between the IGH CDR3 and the epitope, and diagnosing the subject with the autoimmune disease when the CS score is increased relative to a control subject.

In some aspects, disclosed herein is a method of monitoring a subject with an autoimmune disease, the method comprising collecting a first sample from the subject, identifying one or more immunoglobulin heavy chain (IGH) complementarity determining regions (CDR) 3 within the sample, determining a first complementarity score (CS) between the IGH CDR3 and an epitope of the autoimmune disease, wherein the CS is based on electrostatic and hydrophobic interactions between the IGH CDR3 and the epitope, diagnosing the subject with the autoimmune disease when the CS score is increased relative to a control subject, collecting at least one additional sample from the subject at least 14 days after the first sample, determining a second CS between the IGH CDR3 and the epitope of the autoimmune disease, and determining the progression of the autoimmune disease within the subject.

In some aspects, disclosed herein is a method of screening for epitopes of an autoimmune disease, the method comprising collecting a sample from the subject, identifying one or more immunoglobulin heavy chain (IGH) complementarity determining regions (CDR) 3 within the sample, screening the one or more IGH CDR3s against one or more epitopes of an antigen protein associated with the autoimmune disease, and identifying the one or more epitopes of the antigen protein when a complementarity score (CS) between the IGH CDR3 and the epitope is 6 or more, wherein the CS is based on electrostatic and hydrophobic interactions between the IGH CDR3 and the epitope.

Electrostatic interactions refer to the forces of attraction or repulsion between charged particles, such as for example charged amino acids (such as, for example positively charged lysine (Lys), Arginine (Arg), and Histidine (His); and negatively charged aspartic acid (Asp) and glutamic acid (Glu)), wherein oppositely charged particles are attracted and identically charged particles repel from each other. Examples of electrostatic interactions include, but are not limited to hydrogen bonding, base pairing between nucleotides in a DNA double helix, steric hindrance, and protein folding and binding.

Hydrophobic interactions refer to forces of attraction or repulsion that occur when non-polar substances cluster together while repelling water or aqueous substances. Said interactions occur because hydrophobic substances, such as fats and oils, have low solubility in water and are non-polar. Examples of hydrophobic substances include, but are not limited to fat molecules (such as, for example, short, medium and long chain carbon molecules), cholesterol, and some vitamins.

As used herein, “autoimmune diseases” refer to a group of diseases and/or conditions that occur when the body's immune system mistakenly attacks healthy tissues, organs, and cells. The method of any preceding aspect discloses autoimmune diseases including, but not limited to multiple sclerosis, celiac disease, type 1 diabetes, rheumatoid arthritis, systemic lupus erythematosus, psoriasis, scleroderma, inflammatory bowel disease (including, but not limited to Crohn's disease), Graves' disease, Guillain-Barre Syndrome, Chronic inflammatory demyelinating polyneuropathy. Myasthenia gravis, vasculitis, or any combination thereof.

In some embodiments, the subject is administered the therapeutic agent when the CS score is 6 or more. In some embodiments, the subject is administered the therapeutic agent when the CS score is 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, or more. In some embodiments, the subject is administered the therapeutic agent when the IGH CDR3 and the epitope interacts more than 60 times. In some embodiments, the subject is administered the therapeutic agent when the IGH CDR3 and epitope interact 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more times.

In some embodiments, the epitope comprises a whole antigen peptide. In some embodiments, the epitope comprises a partial peptide. As used herein, a “partial peptide” or “a part of a whole” refers to a fragment of the whole antigen peptide, wherein the fragment can be 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the whole antigen peptide.

In some embodiments, the method of any preceding aspect comprises combining the CS scoring disclosed herein with one or more diagnostic tests used to diagnosis and/or monitor an autoimmune disease. In some embodiments, the one or more diagnostic tests include, but are not limited to blood tests (such as, for example an autoantibody screening, an antinuclear antibody test (ANA), a complete blood count (CBC) test, an erythrocyte sedimentation rate (ESR), a comprehensive metabolic panel, a C-reactive protein (CRP) test, or an urinalysis), and/or an imaging modality (such as, for example ultrasound, computed tomography (CT), single photon emission computed tomography, magnetic resonance imaging (MRI), and positron emission tomography (PET)).

In some embodiments, the therapeutic agent comprises an immunotherapeutic agent (including, but not limited to a monoclonal antibody, a CAR T cell therapy, and immune checkpoint inhibitors (including, but not limited to pembrolizumab, nivolumab, and ipilimumab)), a muscle relaxant agent (including, but not limited to diazepam, baclofen, tizanidine, gabapentin, and pregabalin), an analgesic (including, but not limited to ibuprofen, naproxen, and meloxicam), an anti-inflammatory agent (including, but not limited to aspirin, ibuprofen, ketoprofen, naproxen, steroids, glucocorticoids (including, but not limited to betamethasone, budesonide, dexamethasone, hydrocortisone, hydrocortisone acetate, methylprednisolone, prednisolone, prednisone, and triamcinolone), methotrexate, sulfasalazine, lefunomide, anti-Tumor Necrosis Factor (TNF) medications, cyclophosphamide, and mycophenolate), a plasma composition (including, but not limited to therapeutic plasma exchange (TPE) and platelet rich plasma (PRP) injections), a cell-based composition (including, but not limited to CAR-T cell therapies and hematopoietic stem cell transplantation (HSCT), or a combination thereof.

In some embodiments, the sample is a blood sample, a plasma sample, a urine sample, a fecal sample, and any other bodily fluids.

### Computer-Implemented Methods

In some aspects, disclosed herein is a computer-implemented method comprising obtaining or determining, by at least one processor, an immune repertoire for a subject's blood sample, programmatically identifying, by the at least one processor, one or more candidate epitopes corresponding with at least one known or unknown autoimmune disease, by using at least one chemical complementarity algorithm to determine a ratio or value indicating a number of times each of the one or more candidate epitopes complements one of a plurality of amino acids, and determining, by the at least one processor, a disease state or condition of the subject and/or isolating at least one target epitope based, at least in part, on a frequency count and/or degree of correspondence between each respective candidate epitope and respective amino acid.

In some embodiments, the computer-implemented method comprises isolating at least one target epitope and further determining a statistical significance of the at least one target epitope based, at least in part, on a difference in weighted unique residue ratio (WURR) values outside the at least one target epitope relative to one or more control samples.

In some embodiments, identifying the one or more candidate epitopes comprises applying a sliding window analysis with respect to the one or more candidate epitopes and the plurality of amino acids. In some embodiments, the computer-implemented method further comprises generating user interface data (e.g., graphical information, a report) based on the determined disease state or condition of the subject and/or isolated target epitope.

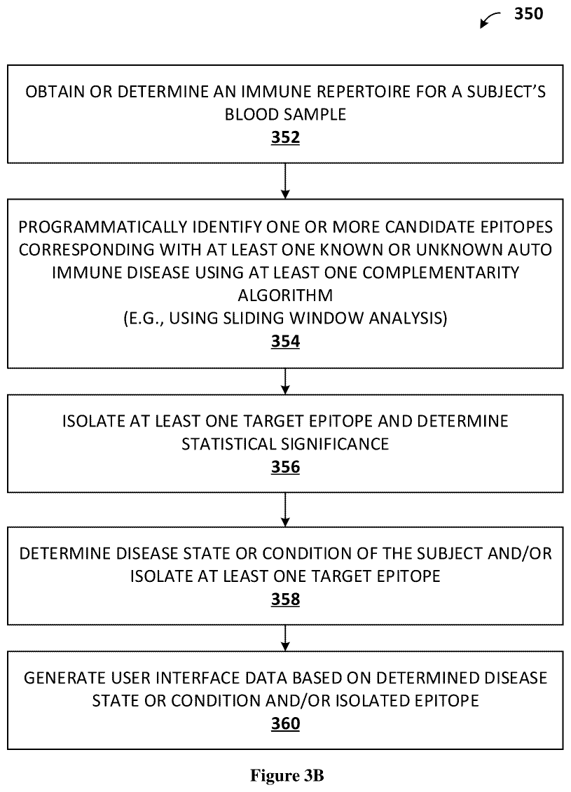

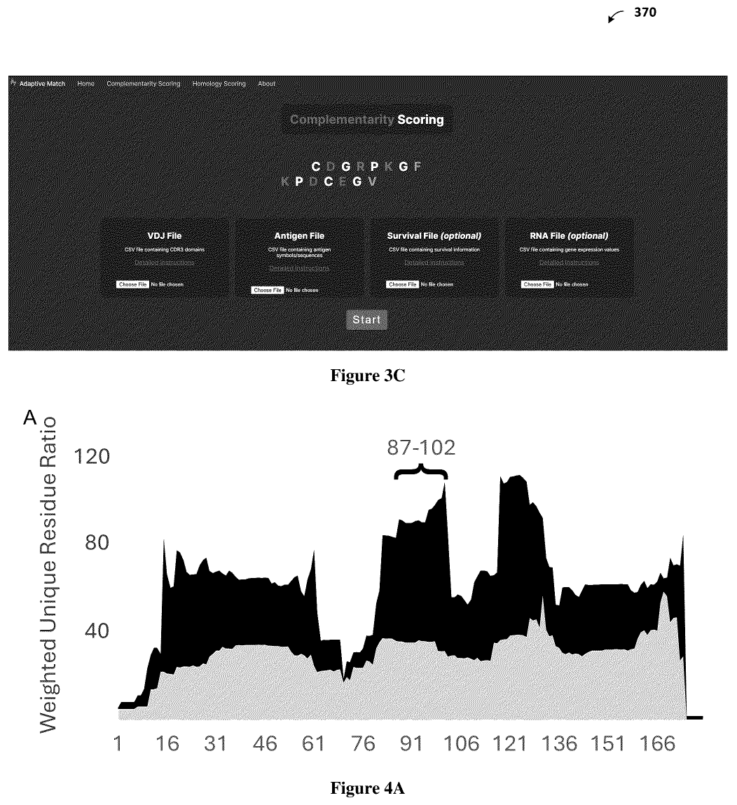

Conventional technologies are not suitable for accurately identifying individuals and/or populations that are at risk for certain autoimmune conditions, for example to confirm a diagnosis and facilitate treatment. FIG. 3A is a flowchart diagram of an example method in accordance with certain embodiments of the present disclosure. FIG. 3B is a flowchart of an example computer-implemented method 350 for determining a disease state or condition of a subject and/or isolating at least one target epitope in accordance with certain embodiments described herein. In some implementations, the methods 300, 350 can be performed by a processing circuitry (for example, but not limited to, an application-specific integrated circuit (ASIC), or a central processing unit (CPU)). In some examples, the processing circuitry may be electrically coupled to and/or in electronic communication with other circuitries of an example computing device, such as, but not limited to, the example computing device 800 described below in connection with FIG. 8. In some examples, embodiments may take the form of a computer program product on a non-transitory computer-readable storage medium storing computer-readable program instruction (e.g., computer software). Any suitable computer-readable storage medium may be utilized, including non-transitory hard disks, CD-ROMs, flash memory, optical storage devices, or magnetic storage devices. This disclosure contemplates that the example operations can be performed using one or more computing devices (e.g., at least the basic configuration illustrated in FIG. 8 by box 802). The example methods 300, 350 can be performed using a computing device/system, as described herein, to facilitate determining a disease state, prognosis, treatment, and/or the like in clinical or laboratory settings. The example computing system can include or host one or more databases, data stores, repositories, and the like (e.g., healthy control databases).

Referring now to FIG. 3A, at step 302, the method 300 includes obtaining immune repertoire base IGH CDR3s for a single sample. At step 304, the method 300 includes retaining CDR3s with minimum chemical complementarity to a selected antigen. At step 306, the method 300 includes determining a total number of copies of all CDR3s complementarity to a given antigen amino acid (AA) residue. At step 308, the method 300 includes determining a number of unique CDR3s complementarity to the given antigen AA residue. At step 310, the method 300 includes determining unique residue ratios (URRs) for all AA residues in the antigen. At step 312, the method 300 includes repeating the preceding steps for all immune repertoire samples and control samples. At step 314, the method 300 includes weighting URRs by relative sample sizes (e.g., to establish weighted unique residue ratios (WURRs)). At step 315, the method 300 includes generating a graphical guide to comparison of WURRs across the complete antigen length. At step 316, the method 300 includes subtracting sample and control WURRs at each residue. At step 318, the method 300 includes averaging the differences and calculating the standard deviation, for example, to establish high difference WURRs. At step 320, the method 300 includes retaining AA residues with high difference WURR values. At step 322, the method 300 includes isolating consecutive AA residues as epitope candidates.

Referring now to FIG. 3B, at step/operation 352, the method 350 includes obtaining or determining (e.g., using the computing device 800 illustrated in FIG. 8) an immune repertoire for a subject's blood sample.

At step/operation 354, the method 350 includes programmatically identifying, by the at least one processor, one or more candidate epitopes corresponding with at least one known or unknown autoimmune disease, by using at least one chemical complementarity algorithm to determine a ratio or value indicating a number of times each of the one or more candidate epitopes complements one of a plurality of amino acids. In some implementations, step/operation 354 includes determining a complementarity score (CS) between the IGH CDR3 and an epitope of the autoimmune disease, wherein the CS is based on electrostatic and hydrophobic interactions between the IGH CDR3 and the epitope. In some implementations, identifying the one or more candidate epitopes comprises applying a sliding window analysis with respect to the one or more candidate epitopes and the plurality of amino acids.

Optionally, at step/operation 356, the method 350 includes isolating at least one target epitope and further determining a statistical significance of the at least one target epitope based, at least in part, on a difference in weighted unique residue ratio (WURR) values outside the at least one target epitope relative to one or more control samples (e.g., obtained from one or more healthy control databases). In some implementations, each ratio or value is weighted and/or averaged based, at least in part, on a size of each candidate epitope in the immune repertoire sample.

At step/operation 358, the method 350 includes determining a disease state or condition of the subject, determining a likelihood that a subject will develop a particular disease or condition (e.g., multiple sclerosis), and/or isolating at least one target epitope based, at least in part, on a frequency count and/or degree of correspondence between each respective candidate epitope and respective amino acid. Additionally, in some implementations, the disease state and/or treatment can be determined using a machine learning model. In some implementations, the method includes determining a prognosis for the subject and/or determining a response to treatment for the subject. Additionally, the method can include providing a determination of minimal or measurable residual disease for the subject or providing a treatment to the subject. Embodiments of the present disclosure contemplate using artificial intelligence and machine learning techniques to at least partially perform the example methods 300, 350. Such techniques can include supervised, semi-supervised, and unsupervised learning models. In a supervised learning model, the model learns a function that maps an input (also known as feature or features) to an output (also known as target or targets) during training with a labeled data set (or dataset). In an unsupervised learning model, the model learns patterns (e.g., structure, distribution, etc.) within an unlabeled data set. In a semi-supervised model, the model learns a function that maps an input (also known as feature or features) to an output (also known as target or target) during training with both labeled and unlabeled data.

At step/operation 360, the method 350 includes generating user interface data (e.g., graphical information, a report) based on the determined disease state or condition of the subject and/or isolated target epitope. Step/operation 360 can include generating and/or outputting a report including data relating to the one or more candidate epitopes, at least one target epitope, and/or the subject's disease state. Alternatively or additionally, the method optionally further includes generating display data for the report. Alternatively or additionally, the method optionally further includes transmitting the report over a network. This disclosure contemplates that operations related to generation of the report can be performed using one or more computing devices (e.g., at least the basic configuration illustrated in FIG. 8 by box 802).

In some implementations, the method optionally further includes, in response to detecting a particular disease state in the subject, providing a diagnosis for the subject. In some embodiments, the data described above are used in combination with other test results (e.g., clinical evaluation) to make the diagnosis. Additionally, the method optionally further includes, in response to detecting a disease state, providing a prognosis for the subject. Alternatively or additionally, the method optionally further includes recommending a treatment for the subject. Treatment approaches can vary depending on the specific disease state, progression, and patient factors. This disclosure contemplates that the operations related to providing diagnosis, prognosis, and/or treatment options can be performed using one or more computing devices (e.g., at least the basic configuration illustrated in FIG. 8 by box 802). Optionally, in some implementations, the method further includes administering the recommended treatment or therapeutic agent to the subject.

Referring now to FIG. 3C, an operational example depicting a user interface 370 that may be generated based at least in part on the above-described operations in FIG. 3B is provided. The computing device 800 may generate and output the user interface data for presentation via the user interface 370. As depicted in FIG. 3C, the user interface 370 allows a user to upload data (as shown, CDR3 domains, antigen symbols/sequences, survival information, and/or gene expression values), that can be used to at least partially perform the methods 200, 350 described above in connection with FIGS. 3A and 3B. The user interface 370 can include various additional features and functionalities for accessing, and/or viewing user interface data. The user interface 370 can also comprise messages to an end-user in the form of banners, headers, notifications, and/or the like. As will be recognized, the described elements are provided for illustrative purposes and are not to be construed as limiting the user interface in any way.

### Computer Systems and Devices

In some aspects, disclosed herein is a system comprising at least one processor and a memory operably coupled to the at least one processor, wherein the memory has computer executable instructions stored thereon that, when executed by the at least one processor, cause the at least one processor to obtain or determine an immune repertoire for a subject's blood sample, programmatically identify one or more candidate epitopes corresponding with at least one known or unknown autoimmune disease, by using at least one chemical complementarity algorithm to determine a ratio or value indicating a number of times each of the one or more candidate epitopes complements one of a plurality of amino acids, determine a disease state or condition of the subject and/or isolating at least one target epitope based, at least in part, on a frequency count and/or degree of correspondence between each respective candidate epitope and respective amino acid.

It should be appreciated that the logical operations described herein with respect to the various figures may be implemented (1) as a sequence of computer-implemented acts or program modules (i.e., software) running on a computing device (e.g., the computing device described in FIG. 8), (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special-purpose digital logic, and any combination thereof. It should also be appreciated that more or fewer operations may be performed than shown in the figures and as described herein. These operations may also be performed in a different order than those described herein.

Referring to FIG. 8, an example computing device 800 upon which embodiments of the present disclosure may be implemented is illustrated. It should be understood that the example computing device 800 is only one example of a suitable computing environment upon which embodiments of the present disclosure may be implemented. Optionally, the computing device 800 can be a well-known computing system including, but not limited to, personal computers, servers, handheld or laptop devices, multiprocessor systems, microprocessor-based systems, personal network computers (PCs), mini-computers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In its most basic configuration, the computing device 800 typically includes at least one processing unit 806 and system memory 804. Depending on the exact configuration and type of computing device, system memory 804 may be volatile (such as random-access memory (RAM)), non-volatile (such as read-only memory (ROM), flash memory, etc.), or some combination of the two. This most basic configuration is illustrated in FIG. 8 by the dashed line 802. The processing unit 806 may be a standard programmable processor that performs arithmetic and logic operations necessary for the operation of the computing device 800. The computing device 800 may also include a bus or other communication mechanism for communicating information among various components of the computing device 800.

Computing device 800 may have additional features/functionality. For example, the computing device 800 may include additional storage such as removable storage 808 and non-removable storage 810 including, but not limited to, magnetic or optical disks or tapes. Computing device 800 may also contain network connection(s) 816 that allow the device to communicate with other devices. Computing device 800 may also have input device(s) 814 such as a keyboard, mouse, touch screen, etc. Output device(s) 812, such as a display, speakers, printer, etc., may also be included. The additional devices may be connected to the bus in order to facilitate communication of data among the components of the computing device 800. All these devices are well-known in the art and need not be discussed at length here.

The processing unit 806 may be configured to execute program code encoded in tangible, computer-readable media. Tangible, computer-readable media refers to any media that is capable of providing data that causes the computing device 800 (i.e., a machine) to operate in a particular fashion. Various computer-readable media may be utilized to provide instructions to the processing unit 806 for execution. Example of tangible, computer-readable media may include but is not limited to, volatile media, non-volatile media, removable media and non-removable media implemented in any method or technology for storage of information such as computer-readable instructions, data structures, program modules or other data. System memory 804, removable storage 808, and non-removable storage 810 are all examples of tangible computer storage media. Examples of tangible, computer-readable recording media include but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 806 may execute program code stored in the system memory 804. For example, the bus may carry data to the system memory 804, from which the processing unit 806 receives and executes instructions. The data received by the system memory 804 may optionally be stored on the removable storage 808 or the non-removable storage 810 before or after execution by the processing unit 806.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, for example, through the use of an application programming interface (API), reusable controls, or the like. Such programs may be implemented in a high-level procedural or object-oriented programming language to communicate with a computer system. However, the program(s) can be implemented in assembly or machine language if desired. In any case, the language may be a compiled or interpreted language, and it may be combined with hardware implementations.

In one embodiment, disclosed herein is a non-transitory computer-readable storage medium comprising instructions that, when executed, cause at least one processor to perform the method of any preceding embodiments.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

The following examples are set forth below to illustrate the compositions, devices, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1: A Computational Approach to Matching Multiple Sclerosis-Related, IGH CDR3s with a MBP Epitope

In multiple sclerosis (MS), T-cell receptors (TCRs) and antibodies specifically target the main structural proteins of myelin, including myelin basic protein (MBP), especially a specific, canonical, immunoglobulin (IG)-targeted MBP epitope. Efficient computational analyses to diagnose or monitor autoimmune conditions, which could have broad applicability in clinical trials or in diagnoses, remains a challenge. As such, it was contemplated that focusing on the immunoglobulin heavy chain (IGH) complementarity determining region-3 (CDR3) amino acid sequences could support the development of an efficient, convenient, and user-friendly approach to detect or assess IGH targets in MS. Thus, a chemical complementarity scoring algorithm, extensively benchmarked in many cancer settings, to assess the combined electrostatic and hydrophobic attractiveness of large numbers of (individual patient) IGH CDR3s and the canonical IG MBP epitope was applied. Samples and controls were filtered to only include CDR3s above a baseline chemical complementarity score. Then, the frequency of each unique IGH CDR3 (with the minimum MBP epitope complementarity) in the MS samples were compared to the chemically complementary to the canonical MBP epitope, was detected in 47 out of 48 MS-control comparisons, in most cases representing a p<0.0001. Thus far, this approach can lead to a user-friendly computational screening tool for patients at risk for developing MS. Additional results indicate that the methodology can also be applied to antigen epitope discovery.

Multiple sclerosis (MS) is an autoimmune condition whereby adaptive immune receptors (IRs) target myelin within the central nervous system, leading to demyelination. Demyelination leads to a variety of clinical neurological manifestations, including optic neuritis, ataxia, fatigue, and sensorimotor defects. T-cell receptor (TCRs) and antibodies specifically target the main structural proteins of myelin: (a) myelin basic protein (MBP), (b) myelin-associated oligodendrocyte basic protein, (c) myelin proteolipid protein, and (d) myelin associated glycoprotein.

Efficient computational analysis to diagnose, monitor, or evaluate autoimmune conditions remain a challenge. As such, we considered the possibility that focusing on the immunoglobulin heavy chain (IGH) complementarity determining region-3 (CDR3), an important segment of the IGH polypeptide for antigen binding, allows for the development of a convenient tool for assessing the IGH impacts in MS. The IGH MBP canonical epitope, considered to represent the main target of IGH is MS, has minor variations in the literature, i.e., as opposed to a consistent, precisely defined amino acid (AA) sequence. The AA sequence that overlaps the sequences identified in most cases, is DENPVVHFFKNIVTPRTPPPSQGK (SEQ ID NO: 1), representing MBP amino acid numbers 83 to 106 in a polypeptide produced by a splice variant referred to as “number 5” or “PO2686-5” in UniProt (www.uniprot.com). Hereinafter, the above indicated MBP AA peptide sequence is referred to as the “canonical epitope”.

Herein, it is contemplated that the MS peptide manifests IGH CDR3s with a significantly increased chemical complementarity to the canonical epitope in comparison to non-MS patients, or that MS IGH CDR3s with higher complementarity to the canonical epitope would occur with an increased frequency in MS patients. To test this, a previously benchmarked chemical complementarity scoring algorithm for simultaneously assessing the combination of electrostatic and hydrophobic attractiveness of IGH CDR3s ad candidate antigens was applied herein. Overall, results indicated a higher frequency of IGH CDR3-canonical epitope pairs with higher chemical complementarity scores (CSs) in MS patients, and identified other, high frequency, high CS, IGH CDR3-candidate epitope pairs specific to MS.

### Methods

Initial IGH CDR3 processing. The IGH CDR3s used herein represent eight MS samples from Palanichamy et al (A. Palanichamy et al. Immunoglobulin class-switched B cells form an active immune axis between CNS and periphery in multiple sclerosis, Sci. Transl. Med. 6(248) 2014; 248ra106, doi.org/10.1126/scitranslmed.3008930. PubMed PMID: 25100740; PubMed Central PMCID: PMCPMC4176763) and eight control samples from Galson et al (J. D. Galson et al. Deep sequencing of B cell receptor repertoires from COVD-19 patients reveals strong convergent immune signatures, Front. Immunol. 11 (2020) 605170. doi.org/10.3389/fimmu.2020.605170. Epub. 20201215.) Each sample was represented by one blood sample. The number of sequences within each sample prior to any processing are available in Table 1. Each set of IGH CDR3 AA sequences was subjected to removal of sequencing artifact symbols that appeared in a subset of the IGH CDR3s. After removal of the symbol, for a given IGH CDR3, the remaining IGH CDR3 AA sequence was, for the purposed of the present disclosure, treated as a complete IGH CDR3 AA sequence. Then, unique IGH CDR3s were counted for each sample, i.e., the frequency of each unique IGH CDR3 for each of the above indicated sixteen (MS sample and control) blood samples were determined. For all samples, unique IGH CDR3 AA sequences were only included in further analysis if occurring at a frequency of ten or more repetitions.

Adaptive Match webtool. To obtain chemical CS for the IGH CDR3 AA sequences and the indicated MBP epitopes, the webtool, adaptivematch.com, which calculates CDR3-candidate epitope CSs based on the algorithm from Chobrutsky et al (B. I. Chobrutsky et al. High-throughput, sliding window algorithm for assessing chemical complementarity between immune receptor CDR3 domains and cancer mutant peptides. TRG-PIK3CA interactions and breast cancer. Mol. Immunol. 135 (2021) 247-253, doi.org/10.1016/j.molimm.2021.02.026. PubMed PMID: 33933816). That is, this webtool applies a step-wise, sliding window alignment approach to assessing the chemical attractiveness of IGH CDR3 AA and candidate epitope sequences. The webtool outputs the highest CS for each IGH CDR3-candidate epitope combination tested. There are instructions for webtool use at Adaptive Match. The webtool outputs what are termed Combo CSs, to reflect assessments of both electrostatic and hydrophobic interactions, with quantitative details in Chobrutsky et al.

Selection of IGH CDR3-MS epitope pairs that represented minimal values, based on their Combo CSs, required from subsequent analyses. Following the chemical complementarity scoring, the matched IGH CDR3-MS epitopes that produced a CS were filtered to identify only IGH CDR3s that began with an AA residue that overlapped MBP AA 65 to MBP AA 106 (with the preceding AA numbers referring to “number 5” or “PO2686-5” in UniProt. That is, the starting AA of the IGH CDR3 had to represent, in the calculation of the Combo CS, contact with (alignment with) MBP AA 65 or an MBP AA after AA 65 through MBP AA 106. Thus, for this above, initial screening, only Combo CSs that represented IGH CDR3s that overlapped the MBP AA 83-MBP AA 106 “window” (representing the canonical epitope), even if that overlap represented only one IGH CDR3 AA, were retained for downstream analyses. Next, the IGH CDR3-canonical epitope Combo CSs were filtered to include only Combo CSs that were scored as 6.0 or above (Table 2).

Student's T-test analysis. As noted in the Discussion, non-equal variance Student's t-tests were used for analyses that were not productive for this report (Tables 4 and 6). After the filtering of samples by the indicated in the section above, the mean value of each sample's IGH CDR3 Combo CSs was calculated (Tables 3 and 5). Each MS sample's Combo CS, respectively, was compared to each control sample's average Combo CSs through a non-equal variance Student's t-test analyses.

Mann-Whitney analysis. The distribution of frequencies of unique IGH CDR3s represented by Combo CSs of 6.0 or above from individual MS and control samples, resulting from the above indicated prescreening process, were compared via Mann-Whitney analysis. Specifically, for each Mann-Whitney analysis, the IGH CDR3s from one MS sample and from one control sample were given ranks, based on the frequency of (repetition) counts of each respective, unique IGH CDR3 (represented by the 6.0 or above Combo CS), from least to greatest utilizing Excel's RANK.AVG function. In this process, several IGH CDR3s representing the lowest frequency would be assigned a rank of 1, followed by a rank of 2, followed by a rank of 3, etc. For any ties of frequency, the ranks were averaged, For example, if the next two CDR2 occurred in the same frequency and represented ranks 4 and 5, the final rank for each would be (4+5)/2=4.5. This process was repeated for all subsequent frequencies. Then, a sum of ranks for the MS sample and a sum of ranks for the control sample were calculated, designated as R1 and R2, respectively. Then, all needed subsequent step to complete the Mann-Whitney analyses were performed. The Mann-Whitney Z score was then used as input for the Microsoft Excel NORM.DIST formula and multiplied by two to generate a two-tailed p-value (Sec Results). An effect size correlation was also calculated. This preceding Mann-Whitney analysis was applied to all sample comparisons. All Mann-Whitney analyses for the canonical epitope following the process are detailed above. (Additional Mann-Whitney analyses are noted in the Results.

Chi-squared proportion analysis. After the Mann-Whitney analysis, the number of unique IGH CDR3s, representing a Combo CS of 6.0 or above, with frequency counts 61 or greater in all MS samples and control samples summed, respectively. The total number of unique IGH CDR3s with a Combo CS of 6.0 or above from MS samples and control samples, respectively, were also summed. Then, utilizing these sums, the proportion of IGH CDR3s with frequencies 61 or greater to total unique IGH CDR3s for MS samples and control samples, respectively, were calculated. The calculated proportion for MS samples was compared to the control samples' proportion utilizing chi-squared analysis of the webtool, MedCalc chi-squared calculator (www.medcalc.org/calc/comparison_of_proportions.php).

### Results

IGH CDR3 frequencies and complementarity scoring with the canonical epitope. To determine whether IGH CDR3s from MS samples represent a greater frequency of IGH CDR3s that have a significantly higher chemical complementarity with the canonical epitope of human MBP, eight MS samples from a single study submitted to the ireceptor.org database was identified, each with a blood sample. The IGH CDR3 AA sequences from these samples were obtained. Each unique IGH CDR3 with a frequency greater than 10, produced by the PCR-based immuno repertoire approach, was evaluated by a previously described chemical complementarity algorithm, termed Combo complementarity scoring, with these evaluations facilitated by adaptivematch.com, which outputs Combo CSs based on the quantification of a combination of hydrophobic and electrostatic attraction. In the first round of assessments of the IGH CDR3s, the MBP AA sequence (representing splice variant 5, also known as P02686-5), was evaluated. Only IGH CDR3s that demonstrated the highest complementarity to an AA sequence that overlapped the non-canonical epitope, defined by residues of MBP 83-MBP 106, were further evaluated. All eight MS samples and eight control samples included IGH CDR3 sequences that demonstrated a positive (non-zero) Combo CS for MBP AA sequences that overlapped the canonical epitope. Herein, it was sought to determine whether the IGH CDR3s with a higher frequency represented the higher Combo CSs, presumably representing an expansion of canonical epitope specific B-cells in the MS samples. Thus, a Combo CS of 6.0 was first established as a minimal CS value. Then, the frequencies of unique IGH CDR3s with a Combo CS of 6 or greater were quantified for the MS and control samples (Table 6). In all MS and control samples, the majority of unique IGH CDR3s with a Combo CS of 6.0 or greater were present in the frequency range of 10-60 repetitions (Table 6). However, the MS samples included more unique IGH CDR3s with a Combo CS of 6.0 or greater in the higher frequency ranges (Table 6). For example, MS samples had an average of 5.5 unique IGH CDR3s in the 211 or greater repetition range, while the controls had an average of 2.6 unique IGH CDR3s in the 211 or greater repetition range.

Mann-Whitney analyses representing the MBP canonical epitope. The presence of greater frequencies of unique IGH CDR3s representing a 6.0 or above Combo CS in the MS samples preliminarily indicated a possible relationship between MS and increased frequency of unique, high Combo CSs for the IGH CDR3s and the canonical epitope. Thus, each MS sample's unique IGH CDR3 frequency distribution was compared to the equivalent distributions represented by each control sample. A series of Mann-Whitney analyses were utilized to compare individual MS samples to individual controls at the canonical epitope on MBP splice variant 5. The Mann-Whitney analyses of MS-5 and MS-7 are listed in Table 7 as examples of the analyses output. Overall, the Mann-Whitney analyses indicated significantly higher frequencies of unique, high Combo CS IGH CDR3s for the MS samples overlapping the canonical epitope (Table 8). The comparison between MS-7 and Control-6 demonstrated that the only incidence of statistical significance where the frequency of unique, high complementarity CDR3s was higher in the control (Table 8).

Proportion analysis representing the canonical epitope. To further evaluate the statistical significance of the relationship between the number of unique, high frequency, high Combo CS IGH CDR3s and MS, a chi-squared proportion analysis was conducted. For MS samples and control samples, a proportion representing the number of unique IGH CDR3s with frequencies of 61 or greater to the total number of unique IGH CDR3s, within all respective samples, was calculated. Note again, these aforementioned IGH CDR3s overlap the canonical epitope in the complementarity scoring analyses. Chi-squared analysis comparison between the MS samples' proportion and the control samples' proportion yielded a highly significant difference (Table 9). This indicates that for MS samples, the number of unique IGH CDR3s with frequency counts 61 or greater make up a larger percentage of all unique IGH CDR3s that demonstrate high complementarity for the MBP canonical epitope in MS, compared to controls.

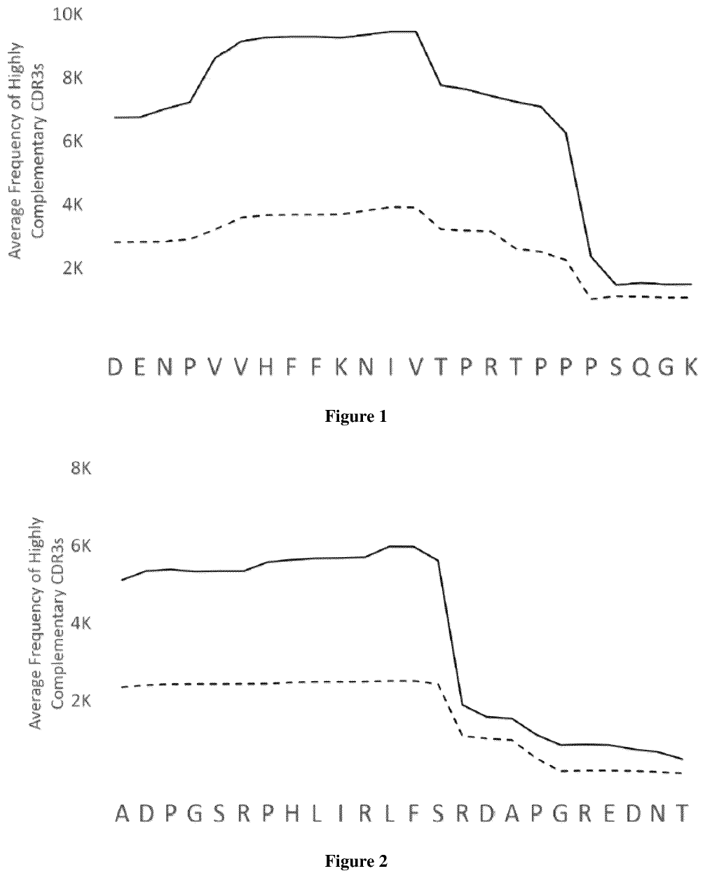

Distinct frequencies of IGH CDR3 interactions with sub-peptides of the canonical epitope. To visualize any variation in the frequency of complementarity within the canonical epitope, the individual residues within the canonical epitope were counted for each instance of IGH CDR3 complementarity, with a Combo CS of 6.0 or above, for each MS sample. The resulting sum from each sample for each residue was the averaged and plotted (FIG. 1; Tables 13 and 14). Within the canonical epitope AA sequence, there is an increase in average number of high Combo CS IGH CDR3s overlapping the Aas beginning with the first valine at position 87 and ending with the valine at position 95. At the start of this peptide, there is an increase in the average number of high Combo CS IGH CDR3s counts to approximately 8600. The increase continues for eight residues, peaking at a count of approximately 9500, before decreasing to 7800 once reaching the threonine at position 96. For roughly six residues, specifically from the proline at residue 97 to the proline at residue 101, the average number gradually declines before dropping dramatically at the last proline at residue 102 (FIG. 1). The increase demonstrated at residues 87 through 95, which corresponds to the peak of high Combo CS IGH CDR3s, are almost an exact match to the dominant T cell and autoantibody epitope described in Wucherpfennig et al and the antibody epitope described in Mameli et al. This process was then repeated for the control samples over the same region for comparison. When compared, the curve representing the controls follows a similar pattern to that of the MS curve until the proline at residue 102, but with many fewer, high Combo CS IGH CDR3s.

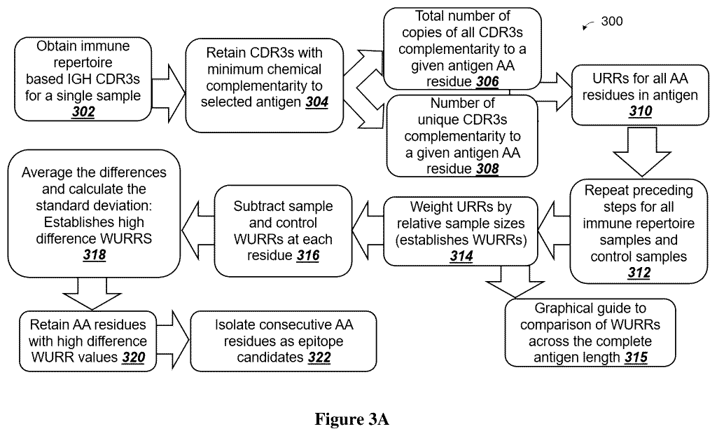

Evaluation of a novel candidate epitope. The algorithm detailed above was repeated for the complete MBP 304 AA sequence (UniProt, P02686-1). All eight MS and control samples included IGH CDR3 sequences that demonstrated a positive Combo CS for IGH CDR3s that overlapped the peptide, ADPGSRPHLIRLFSRDAPGREDNT (SEQ ID NO: 2), in the complete MBP AA sequence. The frequencies of unique IGH CDR3s with a Combo CS of 6.0 or greater that overlapped this novel candidate (non-canonical) epitope were quantified (Table 10). All MS samples demonstrated one or more unique IGH CDR3 with a frequency of 211 or greater at this novel candidate epitope, whereas only half the controls has at least one unique IGH CDR3 with a frequency of 211 or more. (Table 10). The Mann-Whitney series and Chi-squared proportion analysis performed for the canonical epitope were repeated for this candidate epitope. Approximately, 72% of Mann-Whitney analyses comparing each MS and control samples at the candidate epitope demonstrated that MS samples contained high frequencies of unique, high Combo CS IGH CDR3s (Table 11). Chi-squared proportion analysis comparing the proportion of unique, high Combo CS IGH CDR3s with a frequency of 61 or greater to total number of unique, high Combo CS IGH CDR3s for MS and control samples revealed that the MS proportion was significantly greater than the control proportion (Table 12). To visualize any variation in complementarity within the candidate MBP epitope, the graph of FIG. 2 was generated utilizing the same protocol for the generation of FIG. 1. The data utilized to generate FIG. 2 are available in Tables 16 and 16. The MS curve demonstrates a gradual increase from the alanine at position 83 to the phenylalanine at position 95, corresponding to an increase from an average count of approximately 5100-6000 average high Combo CS IGH CDR3s (FIG. 2). The number of average high Combo CS IGH CDR3s then decreases slightly to 5600 at the serine at position 96 before decreasing to less than 2000 (FIG. 2). The curve than maintains a steady decline (FIG. 2). The control curve follows a similar pattern to that of the MS curve, but with many fewer high Combo CS IGH CDR3s until the arginine at position 97.

### Discussion

With the presence of IGH CDR3s that chemically complement the canonical epitope being present in both MS and control patients, it would be expected that IGH CDR3s in MS would possess greater chemical complementarity or that there would be a greater frequency of the chemical complementary IGH CDR3s, or both. MS patients, on average were shown to have more unique, high Combo CS IGH CDR3s overlapping the canonical epitope compared to controls (Table 6). This shows that MS patients have higher serum concentrations of unique IGH CDR3s that demonstrate high complementarity for the canonical epitope, which could represent a B-cell polyclonal expansion. The Mann-Whitney U analysis primarily assess the two distributions to discern whether a significant difference exists between the distributions. Mann-Whitney analyses comparing unique, high complementarity IGH CDR3-canonical epitope pair frequencies of individual MS patients to individual controls demonstrate that for approximately 73% of these comparisons, the MS patients showed an increased frequency of unique, high Combo CS IGH CDR3s (Table 8). More interestingly, all MS patients demonstrated significance in five or more control comparisons, except for the MS-7 case, which only demonstrated significance against two controls (Table 8). However, in the case of one comparison, the comparison of MS-7 to Control-6, the control group has a higher frequency of unique, high complementarity CDR3s (Table 8). The Chi-squared analysis for all controls compared to all MS patients demonstrated that MS patients contained a significantly greater proportion of unique, high complementary, low frequency IGH CDR3s (Table 9). These data further support the literature in that the canonical MBP epitope used for this study is dominant in MS. This congruence demonstrates the likely credibility and utility of the methodology used herein, including reliance on the algorithm of the Adaptive Match web tool.

Concerning the canonical epitope, non-equal variance Student's t-test analysis compared individual average MS patient sample Combo CSs (that were 6.0 and above) to individual control Combo CSs overlapping the epitope to assess if there was a significant difference between groups at the canonical epitope. In approximately 55% of comparisons, there was a significant difference between the groups in favor of controls. Specifically, this was seen most heavily in the comparison of Control-1, Control-2, Control-4, and Control-6 to the MS samples. In approximately 3% of comparisons, there was a significant difference between the groups in favor of the MS samples, specifically in the comparison of MS-1 and MS-3 to Control-7. In the remaining 42%, there was no significant difference between the groups. All average Combo CSs and Student's t-test comparisons can be found in Tables 2, 3, 4, and 5. In sum, the preceding comparisons in the paragraph do not parallel the known biological parameters of the canonical epitope.

Graphical comparisons of the average number of CDR3s complementing each residue in the canonical sequence for MS patients and controls, as seen in FIG. 1, demonstrated higher average numbers of CDR3s along all residues in MS patients, with peaking values from the valine at position 87 to the valine at position 95. This peptide is almost an exact match of the autoantibody epitopes described in Wucherpfennig et al and Mameli et al. This distribution shows that antibodies that specifically complements this peptide are likely more involved in the MS-MBP pathophysiology. Additionally, when visualized, the plot points for the MS and control distributions follow a similar pattern of inflections along with the canonical sequence. Visualizing the novel candidate sequence also demonstrates a similar pattern of inflections between MS patients and controls (FIG. 2). This pattern indicates that MS and control patients have similar chemical IGH CDR3 patterns, but that other key characteristics lead to the development of MS. One contributing factor extensively researched is genetic predisposition. The greatest genetic contributing risk to developing MS is specific variants of human leukocyte antigen II (HLA-II) genes, specifically isotypes HLA-DRB1*15 and HLA-DQB*06:02. Recent genetic mapping has also yielded many other genetic markers that contribute to MS risk.

Mann-Whitney analyses at the novel candidate MBP epitope revealed that MS patients showed an increased frequency of unique, high Combo CS IGH CDR3s (Table 11). Approximately 72% of MS patient to control comparisons were significant, with most MS patients being significant in six or more comparisons, except for MS-7 and MS-8, each of which were significant in four comparisons (Table 11). Chi-squared analysis for all controls compared to all MS patients demonstrated that MS patients contained a significantly greater proportion of unique, high complementary, high frequency IGH CDR3s (that occurred 61 times or more) to total unique, high complementary, IGH CDR3s at the novel candidate epitope (Table 12). The data from the Mann-Whitney and Chi-squared analyses show that MS patients contain more unique, high Combo CS IGH CDR3s that complement the novel candidate region in comparison to control, which shows that the region is an epitope contributing to the autoimmune response.

Graphical comparison of the average number of CDR3s complementing each residue in the novel candidate sequence in FIG. 2 demonstrates a much greater number of average CDR3s from the alanine at position 83 to the serine at position 96 compared to the remaining residues that constitute the originally defined, novel candidate epitope (FIG. 2). This shows that the statistically significant results demonstrated in the Mann-Whitney and Chi-squared analyses are due to high Combo CS, high frequency CDR3s that overlap this specific peptide. Some MS patients, particularly MS-7 at the canonical epitope, demonstrated weak significance in comparison to controls. This is likely due to the multiple etiologies of MS, as MBP is one of four antigens considered relevant for the disease, Future analysis of other antigens of interest, namely myelin-associated oligodendrocyte basic protein, myelin proteolipid protein, and myelin associated glycoprotein, should be considered. Also, this methodology only considers the primary structure of proteins for comparison, excluding the effects of secondary and tertiary structures, along with the addition of protein modifications. In the interest of further evaluating the methodology, the entire methodology was repeated for the canonical sequence of splice variant 5 and the candidate sequence of the complete MBP sequence with the removal of the 6.0 or greater Combo CS filter. For the canonical epitope, removal of the filter only caused a minor decrease in the number of significant comparisons found via Mann-Whitney analyses, specifically a decrease from significance of 73% of comparisons to 69% of comparisons. For the novel candidate epitope, removal of the filter caused a much more substantial drop, specifically from significance of 72% of comparisons to 61% of comparisons. It is worth highlighting that these results demonstrated that this method can readily identify a significant difference between MS and control patients without the Combo CS filter, albeit to a slightly less extent. This implies that the methodology is still useful in identifying high frequency IGH CDR3s that best complement the canonical and candidate sequences in MS patients versus controls with filtering low Combo CS, high frequency CDR3s that likely do not contribute to the disease.

### Conclusion

A chemical complementarity scoring algorithm, supported by a used friendly web tool, can support the distinction of MS patients from controls, based on IGH CDR3 samples. The methods disclosed herein function to distinguish patients representing other autoimmune conditions from healthy controls. Additionally, these methods can be expanded to identify epitopes for other autoimmune conditions.

### Example 2: High-Throughput, Quantitative Approach to Epitope Discovery: A Baseline, IGH-Epitope Interaction Profile that May Represent a Human Predisposition to Autoimmunity

PCR-based, immune repertoire data is commonly used to assess disease features but such data has not yet been used to discover epitopes. This report represents the development of an algorithm that utilizes IGH CDR3s, along with adaptive immune receptor antigen chemical complementarity algorithms, to identify candidate epitopes within known antigens. Thus, a ratio that accounts for the number of times each IGH CDR3 within an immune repertoire sample complements a particular amino acid (AA) residue and the number of unique individual IGH CDR3s that complement that same residue was obtained to develop this IGH CDR3-epitope matching algorithm. Then, these ratios, representing each antigen AA, were weighted by the size of the immune repertoire samples, and the weighted AA ratios for each of the immune repertoires samples was averaged. This process allowed a comparison to a collection of control immune repertoire samples, whereby IGH CDR3s representing high diversity, chemical complementary, and frequency effectively identified epitope candidates. The indicated algorithm was successful in the de novo identification of several known epitopes for multiple sclerosis and celiac disease, respectively; and in the de novo identification of other known epitopes. Also, the above algorithm identified similar patterns of IGH CDR3 diversity and chemical complementarity, but not similar IGH CDR3 frequencies, to known disease epitopes among healthy controls, possibly indicating a basis for a human predisposition to autoimmunity. In conclusion, this strongly indicates the opportunity for computational and user-friendly epitope discovery; and for patient monitoring of adaptive immune receptor-antigen reactivity.

Recently, adaptive immune receptor antigen chemical complementarities, based on computational approaches, have been associated with a large variety of clinical features, especially related to outcomes in the cancer setting. Also, a computational algorithm has recently been developed that was benchmarked with the canonical, multiple sclerosis (MS), immunoglobulin heavy chain (IGH) epitope and that was applied to identify one novel, candidate IGH epitope in the myelin basic protein self-antigen 39644578. However, a reliable, comprehensive approach to antigen epitope discovery via the exploitation of immune repertoire data has yet to be realized. With such an advance, user-friendly, low-level and inexpensive processing, computational algorithms could support in vitro and in vivo experimental approaches, assist in epitope discovery; assist in screening patients at risk of many autoimmune conditions; identify subcategories of patients with autoimmune conditions; and improve our understanding of autoimmune disease origins and pathology in general. In addition, such a comprehensive algorithm, relying on immune repertoire data, could be useful in identifying epitopes in other experimental and patient settings, such as a cancer setting.

Herein, IGH complementarity determining region-(CDR3), amino acid (AA), and immune repertoire data were utilized, as previous work has demonstrated that antibody complementarity can be exclusively dictated by the IGH CDR3 AA sequence. A previously established and extensively benchmarked algorithm for determining AA-CDR3 chemical complementarity based on the combination of electrostatic and hydrophobic interactions, Adaptive Match (adapativematch.com), was also used to determine where, in a given protein-antigen, IGH CDR3s from MS, celiac disease (CD), and control immune repertoire samples, respectively, would best chemically complement a series of known MS and CD antigens. Overall, accessing the immune repertoire IGH CDR3 data, applying the Adaptive Match algorithm, and applying a subsequent series of steps reported here, led to the identification of candidate AA epitopes, within known protein-antigens, that best represented a basic mathematical assessment of IGH CDR3 chemical complementarity and IGH CDR3 diversity and frequency in the immune repertoire collections. Most interestingly, the AA regions of known and newly identified candidate epitopes, within the antigens studied in this report, for both MS and CD, were also apparent via the assessment of IGH CDR3s from healthy controls (albeit without being linked to the high level frequency of IGH CDR3 occurrence seen in the disease states), thereby showing a universal background potential for disease development.

### Methods

IGH CDR3 frequencies in immune repertoire datasets and use of the Adaptive Match web tool. Eight MS (8), eight celiac disease (CD), eight COVID, and eight healthy PCR-based, immune repertoire samples were identified, each representing independent studies submitted to the iReceptor.org database and representing IGH CDR3s. For each study, the IGH CDR3 AA sequences above a frequency of 10 were retained for further analysis. The retained IGH CDR3 sequences were then paired with antigen AA sequences for calculation of Combo CSs, which are chemical CSs that factor in both electrostatic and hydrophobic contributions into a final CS using a sliding window (convolution) process that has been extensively described and benchmarked. The calculations of the Combo CSs were facilitated by use of the Adaptive Match web tool at adaptivematch.com. Only IGH CDR3s that produced a Combo CS greater than or equal to 6.0 were retained for further analysis. An example of this can be seen in Table 17 utilizing 20 IGH CDR3s from MS-8 as the sample and MBP isoform 5 (Uniprot P02686-5) as the antigen. These steps are also summarized in FIGS. 3A and 3B.

Establishing the unique residue ratios (URRs). To determine a here defined, URR, for each amino acid (AA) residue of the antigenic sequence, the IGH CDR3 frequency count was obtained from the sequence files representing each immune repertoire sample, for each unique IGH CDR3s in those files. Then, the CDR3-antigen AA alignment from the Adaptive Match output that produced the Combo CSs (>=6.0) was obtained for each unique IGH CDR3. From this IGH CDR3-antigen AA alignment, the individual AA residues that constituted the overlap, or chemical complementarity, for each unique IGH CDR3. The IGH CDR3 frequency count of each IGH CDR3 with complementarity to an individual residue were then summed. For example, if one AA of a given IGH CDR3 overlapped an antigenic, AA residue, and that IGH CDR3 was repeated in the original immune repertoire file from iReceptor.org 1000 times, then the indicated antigenic AA residue was given a value of 1000. This assessment was repeated for each unique CDR3 that overlapped the indicated AA residue. For example, if the Adaptive Match output provided for three IGH CDR3s that overlapped, or aligned, with that AA residue, producing IGH CDR3-antigen fragment CSs of >=6.0, and the three distinct IGH CDR3s were repeated 1000, 400, and 100 times, respectively, the indicated AA residue would have a tentative value of 1500. This value is herein referred to as the IGH CDR3-antigen AA residue frequency count. This IGH CDR3-antigen AA residue frequency count was then divided by the total number of unique IGH CDR3s that demonstrated chemical complementarity to that indicated residue, thus giving the URR value for that residue. In the above example, the URR would be 1500 divided by 3, yielding a URR value of 500. Thus, by dividing the IGH CDR3-antigen AA residue frequency count by the total number of unique IGH CDR3s overlapping that AA residue, there is a process of normalizing the IGH CDR3 repetitions. For example, if one AA residue overlapped 50 different, unique IGH CDR3s, but only one of those IGH CDR3s was significantly amplified, that amplification would have less value in the subsequent analyses than if 50 IGH CDR3s overlapped a given AA residue and each of those 50 IGH CDR3s were significantly amplified in the original immune repertoire file. The URR calculation is exemplified in Table 18 using selected IGH CDR3s from an MS sample (MS-8) when MBP isoform 5 (Uniprot P02686-5) was used as the antigen. All URR calculations required for this report are available in the supporting online material (SOM).

Establishing the weighted unique residue ratios (WURRs), i.e., weighting by the immune repertoire sample size. To account for the variability in the CDR3 sample size for each PCR-based, immune repertoire sample within a given study, each URR was multiplied by a fraction that represented the number of IGH CDR3s in the sample in which the URR was derived, divided by the total number of IGH CDR3s from all samples used from a particular study. In particular, the WURR represents the final average value after each of the URRs from each of the immune repertoire samples in the study have been individually weighted as described above (Table 19). Note, all WURR calculations for this study are available in the SOM. These steps can be visualized in FIG. 3A and FIG. 3B. To elucidate differences in the WURR values for each residue of an antigen, the WURR values, for each residue, were plotted for the length of the antigen. Both a sample of interest, i.e., an experimental sample, and control sample (represented by WURR values that in turn were generated with IGH CDR3s from disease states or healthy controls with no known connection to the experimental sample) were plotted in the same figure, so that the differences in the overall WURR value distributions could be readily appreciated.

Isolating a potential epitope candidate. For a given comparison group (sample and control), the WURR value for each residue was subtracted, giving a difference value for each residue. The pool of differences along the length of the antigenic sequence were then averaged and the standard deviation of this pool was then found. For those residues who had a difference of at least one standard deviation greater than the average, they were considered residues of interest. For those residues of interest that were continuous with another residue of interest, they were considered a candidate epitope for as long as the residues of interest remained continuous. For isolated residues of interest, i.e. ones that were not continuous with any other residue of interest, they were discarded from further analysis. These steps can be visualized in FIG. 3C.

Statistical significance of a potential epitope candidate's WURR difference when compared to control. For each potential epitope candidate, the difference in WURR values at each residue used to generate the candidate epitope were extracted from the pool of total WURR differences. The average of this group of differences was then found. The average of the remaining differences in WURR values at each residue, i.e. outside of the candidate epitope in question, was then found as well. A heteroscedastic T-test was then performed on these groups to establish statistical significance of these potential epitope sequences from the rest of the antigen.

Combo CS evaluation and statistical significance testing when compared to sample. Using the collection of sample IGH CDR3s and their associated Combo CSs, the sequence of the candidate epitope under evaluation was matched against the antigenic sequence in which each IGH CDR3 best complemented. Any IGH CDR3s in which the potential epitope sequence exactly or internally matched had their associated Combo CSs isolated for further analysis. Note, no IGH CDR3s that only partially overlapped the AA region were used for this step. The isolated Combo CSs from each sample were then pooled and an average Combo CSs was found for the potential epitope candidate. Any IGH CDR3s in which the potential epitope sequence did not match had their associated Combo CSs pooled as well, with the average of this pool also being found. Those candidate epitopes with matching IGH CDR3s with average Combo CSs greater than the Combo CSs of IGH CDR3s that did not match the candidate epitopes' sequence continued on to further analysis. Those that were less than average were discarded. Using these two pools of Combo CSs, a heteroscedastic T-test was performed to establish statistical significance of the increased Combo CS values for the candidate epitope sequence when compared to all other residues within the antigen.

IEDB partial matching protocol. For those candidate epitopes that were found to be significant, their sequences were partially matched against the sequences provided in the IEDB for their specific antigen that they were derived, unless otherwise noted. To do this, the database for a specific antigen was downloaded from the IEDB using the following parameters: (a) epitope structure→any; (b) epitope source, organism→Homo sapiens (human) (ID: 9606, human); (c) epitope source, antigen→antigen of interest; (d) host→any; (e) assay→B cell→outcome: positive; (f) MHC restriction→any; (g) disease→any. Note, these parameters were slightly tweaked for EBV Nuclear Antigen 1 analysis: (a) epitope structure→any; (b) epitope source, antigen→Epstein-Barr nuclear antigen 1 [P03211] (EBNA-1); (c) host→human; (d) assay→B cell→outcome: positive; (e) MHC restriction→any; (f) disease→any.

Once the database was downloaded, the sequence undergoing analysis was compared individually to each entry in the database by assigning a numeric value to each residue based on its position within the antigen. Then, each residue of the database sequence was also assigned a value based on its position within the antigen. Note, variability of the sequences due to biochemical means was also accounted for by allowing one AA to be incorrect for every ten AAs in the sequence being compared when assigning numeric values. For example, if the antigenic sequence was TQDENPVVHF (SEQ ID NO: 3) at positions 81-90, but the sequence in question was TQDQNPVVHF (SEQ ID NO: 4), this sequence would be labeled as positions 81-90, even though the fourth residue differs. In cases where the investigated antigen was not available in the IEDB, the closest match was used. In any case where a database entry was not found in the investigated antigen due to the difference, that entry was removed from analysis. Both the forward direction (candidate epitope sequence against database entry sequence) and reverse direction (database entry sequence against candidate epitope sequence) were assessed and the percentage in which they matched, based on the assigned numeric values, was generated. Any entry that generated a percentage above 10% in both directions were counted towards the total number of partially matched entries for that candidate epitope. For example, a candidate epitope sequence of GAEGQRPGFGYGG (SEQ ID NO: 5) and a database entry sequence of WGAEGQKPGFGYGG (SEQ ID NO: 6) would yield approximately a 92% match in the forward direction and an 86% match in the reverse direction. The 92% match in the forward direction is due to a variation in the sixth residue (R) of the candidate epitope and seventh (K) of the database entry sequence, so 12 out of the 13 residues match. The 86% match in the reverse direction is due to the above variation and the addition of an amino acid at the beginning of the database entry sequence (W), so 12 out of the 14 residues match. Given that both directions yielded a percentage above 10%, this was considered a partial match.

Chi-square analysis of IEDB matching. Once all IEDB partial matching results were collected, the total number of unique sequences matched against the IEDB from all antigens were counted, which may vary from the total number of sequences matched against the IEDB due to some antigens being analyzed against multiple controls. The number of matches of these unique sequences were recorded, as well as the number of unique sequences that did not match. Using these two datapoints, a chi-square proportion analysis was performed using the “comparison of proportions calculator” webtool from MedCalc (www.medcalc.org/calc/comparison_of_proportions.php) to determine if this relationship was significant.

Flowchart of the above algorithm. A summary of these methods can be found in FIGS. 3A and 3B, which allow for a more granular breakdown of the algorithm described above.

Summary of application of above protocols. The above processes that lead to the generation of WURRs for each AA in the antigens, in turn based on the adaptivematch.com outputs and frequency counts from the immune repertoire samples, were performed for the following antigens: MBP isoform 5 (Uniprot P02686-5), proteolipid protein (Uniprot P60201), alpha/beta-gliadin MM1 (Uniprot P18573), prolamin (Uniprot D2T2K3), gamma gliadin (P08453), and EBV Nuclear Antigen 1 (Uniprot P03211) (FIGS. 3A and 3B).

### Results

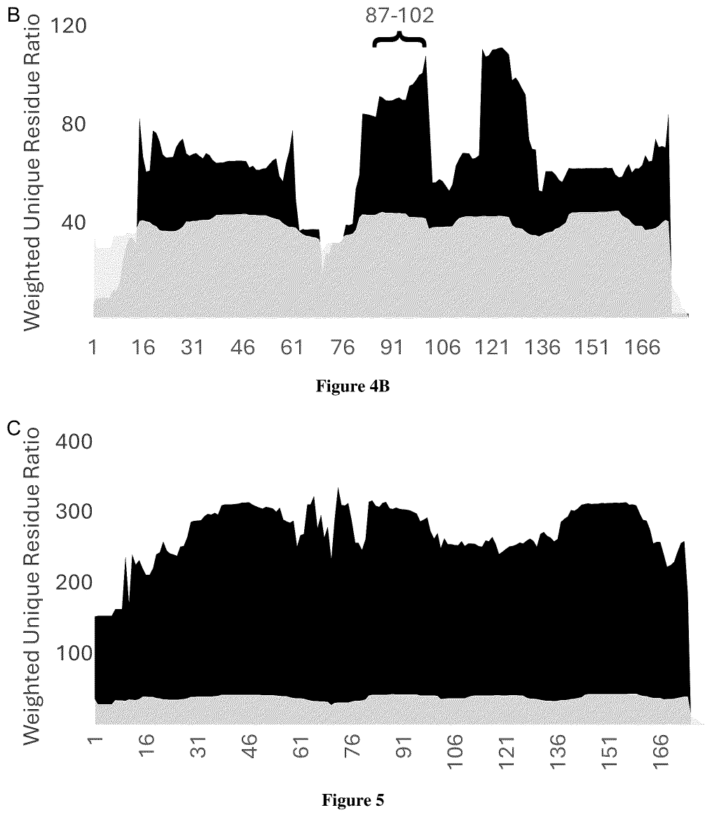

Defining candidate epitopes for MS IGH CDR3s for MBP isoform 5. To determine continuous AA sequences of MBP isoform 5 that represent candidate epitopes for IGH CDR3s that were derived from MS IGH CDR3, immune repertoire samples, the WURR values (representing individual AAs; Methods) based on these MS IGH CDR3s, were compared to the WURR values based on the IGH CDR3s from both the COVID and Healthy immune repertoire samples, i.e., the latter two IGH CDR3 datasets were used as negative controls. Continuous AA sequences with highly different WURR values (i.e., representing one SD unit above the average WURR differences), for the MS IGH CDR3s versus control IGH CDR3s, herein referred to as the “high difference” WURRs, were recorded as described (Methods) (FIGS. 4A and 4B). These continuous AA sequences, of which there were several, with high difference WURRs underwent a heteroscedastic T-test (Methods), whereby the continuous AA sequences with the high difference WURRs were compared to the remaining AA sequence of the MBP isoform 5 (FIGS. 4A and 4B). Notably, this preceding approach identified an AA sequence, VVHFFKNIVTPRTPPP (residues 87-102; SEQ ID NO: 7) from MBP isoform 5 representing the canonical MBP antibody epitope for MS. That is, the canonical epitope was identified by the high difference WURRs for the MS-COVID comparisons and by the high difference WURRs for the MS-Healthy comparisons. One additional candidate epitope, where high difference WURRs were based on the MS-COVID and the MS-Healthy comparisons, respectively, was also detected (Table 20).

Keeping in mind the above high difference WURR approach, a related but distinct approach to identify candidate epitopes for MBP isoform 5 was considered. Thus, Combo CSs for the MS IGH CDR3s that were precisely aligned, from beginning to end AAs, i.e., had the same number of AAs as the candidate epitope, or were completely internally aligned with a candidate epitope AA sequence, that in turn represented high difference WURRs (Methods) for the MS IGH CDR3s versus the control sample (COVID or Healthy) IGH CDR3s, were obtained. (Note, no IGH CDR3s that only partially overlapped the candidate epitope AA sequence representing the high difference WURR values were used for this step (Table 21). The Combo CSs represented by the high difference WURR AA sequences were thus compared to the Combo CSs for all other IGH CDR3 AA sequence alignments for the MBP isoform 5 protein. This comparison allowed an increased emphasis on the distinction between high difference WURR AA sequences from other AA sequences as being due to chemical complementarity between IGH CDR3s and candidate epitopes. Thus, if the high difference WURR AA sequences' IGH CDR3s had an average Combo CS that was above the average Combo CS for the remaining IGH CDR3-pairs (for the MBP isoform 5 AA sequences), then a heteroscedastic T-test was performed to establish statistical significance of the increased Combo CS values for the high difference WURR AA sequence. This approach is herein referred to as the WURR-Combo CS approach. The high difference WURR AA sequences that demonstrated significance via the WURR-Combo CS approach were then compared against the epitope AA sequences of the IEDB, and the number of partial matches against the database entries were obtained via the protocol described in Methods. All AA sequences in MBP isoform 5 identified as indicated using the MS IGH CDR3s demonstrated significance via the T-test (Table 22). The MS-COVID comparison produced 3 candidate epitopes, while the MS-Healthy comparison produced 2 candidate epitopes. Of the MS-COVID comparisons, residues 19-20, 87-102, and 118-130 partially matched 4, 20, and 5 IEDB entries respectively. Of the MS-Healthy comparisons, residues 87-102 and 118-131 partially matched 20 and 5 IEDB entries respectively. The two regions (residues 87-102 and 118-130) are illustrated by the large peaks defined by the indicated residue numbers, in FIGS. 4A and 4B. Notably, the region defined by residues 87-102, representing VVHFFKNIVTPRTPPP (SEQ ID NO: 7), overlaps what is widely considered the canonical epitope for MBP isoform 5.

Note, the above approaches (WURR and WURR-Combo CS) were also applied to the myelin proteolipid protein, with results indicating candidate epitopes for this protein.

Verifying the specificity of the algorithm. To ensure that the high difference WURR values identifying the canonical MS epitope and likely candidate MS epitopes represented an MS specific, algorithm outcome, the same analysis on MBP isoform 5 was done with IGH CDR3s from CD samples. This analysis revealed no regions representing high difference WURRs per the algorithm used above and outlined in Methods (FIGS. 3A and 3B). Although the WURRs based on the IGH CDR3s from the CD immune repertoire samples overall yielded higher values in comparison to the WURRs based on the CDR3s from the Healthy control samples, there were no continuous AA sequences for which the difference between the WURR plots was represented by at least one standard deviation greater than the average WURR value. (As detailed in Methods, one standard deviation unit above the average WURR differences defines the high difference WURRs.)

Defining candidate epitopes for alpha/beta-gliadin MM1. To determine continuous AA sequences that represent candidate epitopes for alpha/beta-gliadin MM1 (Uniprot P18573), first the WURR approach was applied using the WURR values based on the CD IGH CDR3s. These were compared to the WURR values based on the IGH CDR3s from the Healthy immune repertoire samples. These continuous AA sequences, of which there were several, with high difference WURRs underwent a heteroscedastic T-test (Methods), with results indicating four candidate epitopes (Table 23).

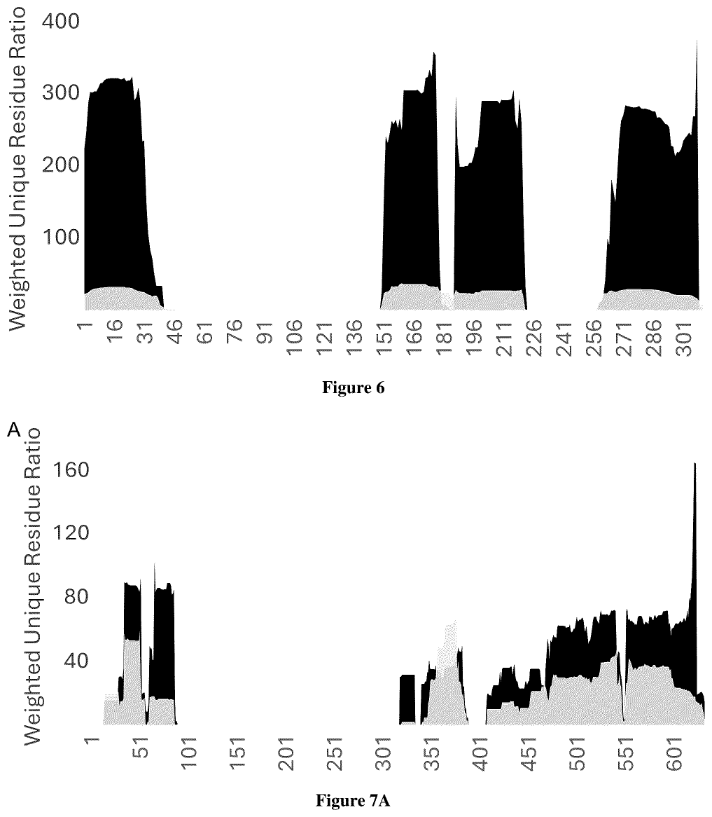

Next, the WURR-Combo CS approach was followed using the CD-COVID and CD-Healthy comparisons. The high difference WURR AA sequences that demonstrated significance via the WURR-Combo CS approach were then compared against the epitope AA sequences of the IEDB, and the number of partial matches against the database entries were obtained via the protocol described in Methods. Five regions of interest were identified, with only four of these being above average combo complementary score and significant (Table 24). These remaining four were partially matched against the IEDB for Tri a 21, as alpha/beta gliadin MM1 is not listed in the database, however, this difference was accounted for in this report as described in Methods. All four of the sequences were found in the IEDB. The sequences found between residues 3-29, 200-217, 270-291, and 306-308 partially matched 13, 18, 17, and 4 IEDB entries, respectively. Notably, the sequence between residues 270-291, LPQFEEIRNLALETLPAMCNVY (SEQ ID NO: 8) contains a region with one amino acid substitution noted by Jain et al. (PMID 38537966) to be one of significant interest, that being LALQTLPAMC (SEQ ID NO: 9). The substitution of Q to E possibly being related to the deamination seen in the celiac disease state (PMID: 30678169). These regions can be seen within the peaks in FIG. 6. Note, the above approaches (WURR and WURR-Combo CS) were also applied to additional antigens related to CD.

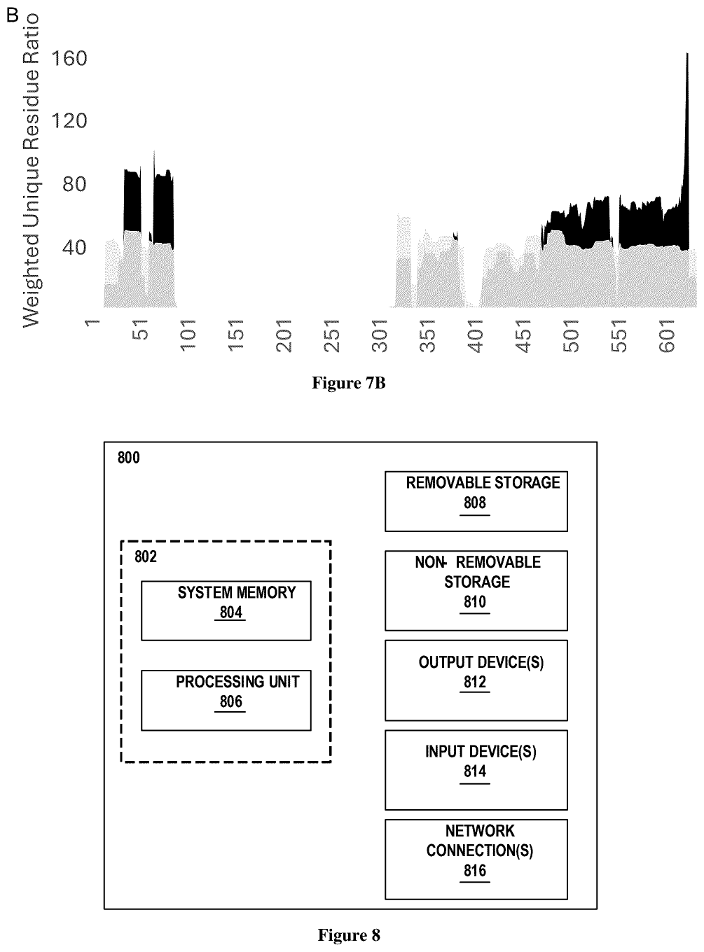

MS-COVID and MS-Healthy WURR comparisons with Epstein Barr Virus (EBV) Nuclear Antigen 1. Due to the relationship between EBV and the potential pathogenesis of MS, both the WURR and WURR-Combo CS approaches were performed using EBV Nuclear Antigen 1 (Uniprot P03211) for the MS-COVID and MS-Healthy comparisons (Table 25). The MS-COVID comparison produced one candidate epitope via the WURR-Combo CS approach that was statistically significant (FIG. 7A). The MS-Healthy comparison produced six significant sequences that all partially matched multiple IEDB entries (FIG. 7B).

Chi-square analysis of IEDB matching results. To determine whether the proportion of all of the candidate epitopes identified above (Tables 20 and 23) with at least one partially matched IEDB entry to the proportion of those with no partial matches to any IEDB entry for the antigen's respective database was significant, a chi-square proportion analysis was performed. The result of this analysis was a significant p value of <0.0001 (Table 25; Methods).

### Discussion

The present disclosure implements a ratio that accounts for the number of times each IGH CDR3 within a sample complements a particular residue of an epitope to the number of unique, individual IGH CDR3s that complement the same residue.

Using immune repertoire data to identify a set of IGH CDR3s that complement a disease specific antigen, certain individual residues of the antigenic sequence are represented by more IGH CDR3s over regions that contain epitopes. Moreover, by creating a ratio that accounts for the number of times each IGH CDR3 within a sample complements a particular residue and the number of unique, individual IGH CDR3s that complement the same residue, amino acid residues of importance should be identifiable once weighted by sample size and thus would allow for isolation of candidate epitopes when compared to control states. From this research, candidate epitopes were able to be isolated by these means. Heteroscedastic T-tests were employed in two separate instances to assess the significance of the candidate epitopes isolated, that of the Combo CS scores and the WURR values associated with the candidates. The candidate epitopes isolated were further supported by entries in the IEDB that reflect previous research as well as literature that supports that these regions isolated are implicated in MS and CD. These points enhance the credibility of the methodology itself in identifying known epitopes as well as its ability to identify candidate epitopes where one has not been well researched by previous methods.

Summary of results for MS comparisons. MBP isoform 5 was utilized as the antigen of interest for MS samples since this antigen is well documented as one of interest in the literature. This method was able to isolate 2 candidate epitopes, both with a high degree of significance with both T-tests (Tables 20 and 22). Notably, the candidate epitope isolated between residues 87-102 is considered the canonical epitope. These data are also represented in FIGS. 4A and 4B. Further analysis was done utilizing the MBP isoform 5 antigen with CD IGH CDR3 samples, finding no candidate epitopes, showing that this algorithm is specific to the disease state being tested. This finding is illustrated in FIG. 5, with the CD sample consistently demonstrating higher WURR values per residue, but never demonstrating a region that is distinctly different from the rest of the peptide, showing the higher values are due to a nonspecific autoimmune state seen in the disease that does not interact with MBP isoform 5.

Another antigen of interest believed to be involved in the pathogenesis of MS is PLP. When used as the antigen of interest in this analysis, two sequences of significance were found (Table 23). One of these sequences was not represented in the IEDB, possibly implying that this is a candidate epitope that has yet to be discovered by any other means (Table 24).

Investigation of EBV Nuclear Antigen I was also performed due to the literature supporting the influence of EBV infection with MS. This analysis noted 2 and 5 candidate epitopes that demonstrated significance when compared to COVID and Healthy controls. However, most notably from this analysis, the MS-COVID and MS-Healthy comparison (FIGS. 7A and 7B) demonstrate higher WURR values from the MS samples for the majority of regions of the EBV Nuclear Antigen 1 sequence, consistent with a potential link between the two disease states.

Summary of results for CD comparisons. Several antigens of interest in CD were investigated for the CD samples, specifically alpha/beta gliadin MM1, prolamin, and gamma gliadin. The analysis of alpha/beta gliadin MM1 produced 3 candidate epitopes found to be significant in both analyses that were supported by entries in the IEDB (Tables 23 and 24). These results are well visualized in FIG. 6. The analysis of prolamin produced 2 sequences of indeterminate significance, due to the limitations expressed below, that were represented in the IEDB (Tables 23 and 24). Finally, the analysis of gamma gliadin produced 3 candidate epitopes, one of which being of indeterminant significance that does appear in the IEDB, due to its length (Tables 23 and 24). Additionally, one of the found sequences was not represented in the IEDB, suggesting another candidate epitope that may have not been discovered yet by other means. Notably, the candidate epitope seen in the analysis of alpha/beta gliadin MM1, between residues 270-291, and in the analysis of gamma gliadin, between residues 277-328, align with the literature as a significant epitope of interest (PMID 38537966).

Common background IGH CDR3-epitope interactions in samples studied in this report. Perhaps the most notable result from all these analyses is the trend in which the control plots tend to follow the sample plots. Shown in FIGS. 4A, 4B, 6, 7A, and 7B, the control plots tend to mirror the sample plots. This close resemblance implies that certain regions are more prone to being immunogenic than others, especially over the regions in which candidate epitopes lie. This may be evidence of a baseline auto-immunogenicity seen in all humans. With this apparent baseline, only some individuals develop the disease state in question, possibly due to the influence of HLA types or environmental influences. Overall, results indicated that this algorithm identifies the canonical epitope (MBP splice variant 5, residues 87-102) on MBP (35795217, 9276728, 29428829), along with several other regions of interest on MS and CD antigens. Additionally, these findings are disease specific, as when MS antigens were tested against CD IGH CDR3s, no results were found.

Multiple sclerosis (MS) is an autoimmune condition of great clinical interest. MS is a neurological disease whereby oligodendrocytes, the cells responsible for myelinating the brain and spinal cord axons, are targeted and damaged by T cells and antibodies, resulting in dysfunction of action potential propagation 29763024.MS represents a variety of clinical manifestations, including but not limited to sensory defects, motor defects, ataxia, fatigue, optic neuritis, and internuclear ophthalmoplegia 29763024. The development of MS and the exact mechanism of oligodendrocyte-mediated destruction are not completely understood. Persons with specific HLA-DR mutations, vitamin D deficiency, and previous Epstein-Barr virus (EBV) infection are at increased risk for MS 29763024. Self-antigens targeted in MS are believed to be myelin basic protein (MBP), myelin proteolipid protein (PLP), myelin associated glycoprotein, and myelin-associated oligodendrocyte basic protein. MBP, specifically its fifth splice variant referred to as P02686-5 in Uniprot (unitprot.org), has a canonical epitope that has been extensively verified in the scientific literature.

Celiac disease (CD) is a gastrointestinal disorder where there is breakdown of enterocyte tight junctions in response to gliadin, a protein found in grains (PMC5437500). This leads to an immune response to gliadin proteins which leads to an inflammatory Th1 and Th2 response. This response leads to a clonal expansion of B-cells, leading to anti-gliadin antibodies, as well as anti-tissue-transglutaminase antibodies (PMC5437500). This leads to lethargy, diarrhea, abdominal pain, vomiting, constipation, poor nutrient absorption sequalae (anemias, coagulopathies, osteoporosis, neurological symptoms), and dermatitis herpetiformis (PMC5437500 and 28722929).

Weighted unique residue ratios based on IGH CDR3 samples can be utilized, in combination with the Adaptive Match web tool, to identify candidate epitopes on an antigen of interest for a known disease state when compared to controls. These methods may be utilized to develop individualized therapies and early diagnostic methods and have utility in other biochemical realms. These methods have been able to demonstrate a baseline, IGH-epitope interaction profile that represents a human predisposition to autoimmunity.

It will be apparent to those skilled in the art that various modifications and variations can be made in the present disclosure without departing from the scope or spirit of the invention. Other embodiments of the disclosure will be apparent to those skilled in the art from consideration of the specification and practice of the methods disclosed herein. It is intended that the specification and examples be considered as exemplary only, with a true scope and spirit of the invention being indicated by the following claims.

### TABLES

TABLE 1
Number of sequences within each sample prior to any processing.

| | Original numbers of |
|---|---|
| Sample | recombination reads |
| MS 1 | 97286 |
| MS 2 | 141696 |
| MS 3 | 175140 |
| MS 4 | 161449 |
| MS 5 | 162675 |
| MS 6 | 177810 |
| MS 7 | 148142 |
| MS 8 | 117778 |
| MS Average | 147747 |
| Control 1 | 298430 |
| Control 2 | 294034 |
| Control 3 | 273730 |
| Control 4 | 85917 |
| Control 5 | 82981 |
| Control 6 | 69903 |
| Control 7 | 64415 |
| Control 8 | 57819 |
| Control Average | 153404 |

TABLE 2
Average High Combo CSs at Canonical Epitope.

| Sample | Combo CSs |
|---|---|
| MS 1 | 8.496911393 |
| MS 2 | 8.10634551 |
| MS 3 | 8.56406234 |
| MS 4 | 8.371622555 |
| MS 5 | 8.296812363 |
| MS 6 | 8.146995355 |
| MS 7 | 8.003844564 |
| MS 8 | 8.155496726 |
| Control 1 | 8.672486988 |
| Control 2 | 8.984971291 |
| Control 3 | 8.37956456 |
| Control 4 | 9.25072167 |
| Control 5 | 8.275418824 |
| Control 6 | 9.470546982 |
| Control 7 | 7.786093445 |
| Control 8 | 8.547269814 |

TABLE 3
Blood Combo CS Comparison Unequal Variance at Canonical Epitope with 6.0 or greater Combo CS filter.

| | MS 1 | MS 2 | MS 3 | MS 4 | MS 5 | MS 6 | MS 7 | MS 8 |
|---|---|---|---|---|---|---|---|---|
| Control | 0.370063 | 2.23E−05 | 0.511975 | 0.029509 | 0.023697 | 1.79E−05 | 1.36E−07 | 0.007854 |
| 1 | | | | | | | | |
| Control | 0.016596 | 1.04E−09 | 0.015275 | 3.26E−05 | 8.16E−05 | 3.79E−10 | 1.24E−12 | 4.43E−05 |
| 2 | | | | | | | | |
| Control | 0.538903 | 0.029393 | 0.248705 | 0.951669 | 0.6045 | 0.041094 | 0.00148 | 0.233303 |
| 3 | | | | | | | | |
| Control | 0.013016 | 5.22E−05 | 0.016018 | 0.001476 | 0.001027 | 7.25E−05 | 1.19E−05 | 0.000373 |
| 4 | | | | | | | | |
| Control | 0.359636 | 0.384434 | 0.186416 | 0.627002 | 0.921346 | 0.492552 | 0.153808 | 0.615799 |
| 5 | | | | | | | | |
| Control | 0.00168 | 2.31E−06 | 0.001802 | 0.000106 | 7.71E−05 | 3.24E−06 | 4.25E−07 | 2.75E−05 |
| 6 | | | | | | | | |
| Control | 0.035032 | 0.286084 | 0.016236 | 0.056835 | 0.108315 | 0.223474 | 0.462092 | 0.263785 |
| 7 | | | | | | | | |
| Control | 0.846938 | 0.046497 | 0.944026 | 0.428539 | 0.296737 | 0.062631 | 0.013354 | 0.131869 |
| 8 | | | | | | | | |

TABLE 4
Average High Combo CSs at Candidate Epitope.

| Sample | Combo CSs |
|---|---|
| MS 1 | 8.248087106 |
| MS 2 | 8.296026493 |
| MS 3 | 8.141187788 |
| MS 4 | 8.315397927 |
| MS 5 | 8.192943634 |
| MS 6 | 8.171721001 |
| MS 7 | 7.872776832 |
| MS 8 | 8.264350159 |
| Control 1 | 8.358416408 |
| Control 2 | 8.669398258 |
| Control 3 | 8.471112284 |
| Control 4 | 8.51895583 |
| Control 5 | 8.125731139 |
| Control 6 | 8.434702075 |
| Control 7 | 8.25769628 |
| Control 8 | 8.309876209 |

TABLE 5
Blood Combo CS Comparison Unequal Variance at Candidate Epitope with 6.0 or greater Combo CS filter.

| | MS 1 | MS 2 | MS 3 | MS 4 | MS 5 | MS 6 | MS 7 | MS 8 |
|---|---|---|---|---|---|---|---|---|
| Control | 0.548429 | 0.651529 | 0.394575 | 0.751574 | 0.336618 | 0.11533 | 5.68E−05 | 0.604748 |
| 1 | | | | | | | | |
| Control | 0.028031 | 0.011003 | 0.045043 | 0.014407 | 0.008108 | 0.000114 | 1.54E−09 | 0.032332 |
| 2 | | | | | | | | |
| Control | 0.228893 | 0.209957 | 0.199334 | 0.257124 | 0.109212 | 0.012903 | 1.07E−06 | 0.258855 |
| 3 | | | | | | | | |
| Control | 0.348468 | 0.396094 | 0.264005 | 0.436018 | 0.248579 | 0.174544 | 0.015049 | 0.375848 |
| 4 | | | | | | | | |
| Control | 0.629187 | 0.445585 | 0.959961 | 0.392974 | 0.783871 | 0.827625 | 0.235461 | 0.582279 |
| 5 | | | | | | | | |
| Control | 0.420991 | 0.483674 | 0.313861 | 0.543442 | 0.278742 | 0.15877 | 0.00385 | 0.45935 |
| 6 | | | | | | | | |
| Control | 0.964126 | 0.827907 | 0.673668 | 0.741078 | 0.750735 | 0.594817 | 0.020762 | 0.974954 |
| 7 | | | | | | | | |
| Control | 0.785811 | 0.942697 | 0.557337 | 0.976947 | 0.592119 | 0.441844 | 0.017746 | 0.840173 |
| 8 | | | | | | | | |

TABLE 6
Number of unique, high Combo CS IGH CDR3s at specific frequency ranges for
each MS and control sample overlapping the canonical epitope. Note, the
average total (starting) number of IGH recombination reads for the MS samples
was 147,747 and for the control samples was 153,404 (Table 1).

| | Number of | Number of | Number of | Number of | Number of |
|---|---|---|---|---|---|
| | unique IGH | unique IGH | unique IGH | unique IGH | unique IGH |
| | CDR3s with | CDR3s with | CDR3s with | CDR3s with | CDR3s with |
| Sample | frequency | frequency | frequency | frequency | frequency |
| number | 10-60 | 61-110 | 111-160 | 161-210 | 211+ |
| MS-1 | 50 | 8 | 5 | 3 | 3 |
| MS-2 | 182 | 16 | 3 | 3 | 7 |
| MS-3 | 61 | 8 | 1 | 4 | 9 |
| MS-4 | 178 | 8 | 5 | 1 | 5 |
| MS-5 | 83 | 17 | 8 | 4 | 12 |
| MS-6 | 288 | 17 | 0 | 2 | 4 |
| MS-7 | 276 | 10 | 2 | 5 | 1 |
| MS-8 | 82 | 5 | 6 | 1 | 3 |
| Control-1 | 262 | 7 | 4 | 0 | 9 |
| Control-2 | 205 | 11 | 3 | 5 | 2 |
| Control-3 | 298 | 9 | 1 | 0 | 4 |
| Control-4 | 38 | 2 | 0 | 0 | 1 |
| Control-5 | 57 | 2 | 1 | 0 | 2 |
| Control-6 | 47 | 3 | 3 | 0 | 3 |
| Control-7 | 31 | 2 | 0 | 0 | 0 |
| Control-8 | 43 | 0 | 0 | 0 | 0 |

TABLE 7
Example MS samples 5 and 7 Mann-Whitney analyses
(versus controls) for the frequencies of the high
Combo CS CDR3s overlapping the canonical epitope.

| | Mann- | | 2 Tailed | Effect |
|---|---|---|---|---|
| MS-5 | Whitney U | Z value | p-value | size |
| Control-1 | 11593.5 | −5.4161425 | 6.0896*10\hat{ }−8 | 0.269 |
| Control-2 | 9765 | −4.69673 | 2.64*10\hat{ }−6 | 0.251 |
| Control-3 | 9291.5 | −8.49841 | 1.92*10\hat{ }−17 | 0.407 |
| Control-4 | 1760 | −2.95074 | 0.00317 | 0.230 |
| Control-5 | 2446 | −4.0428 | 5.28*10\hat{ }−5 | 0.296 |
| Control-6 | 2934.5 | −1.66182 | 0.09655 | 0.124 |
| Control-7 | 1171.5 | −3.77017 | 0.000163 | 0.301 |
| Control-8 | 1370.5 | −4.74714 | 2.06*10\hat{ }−6 | 0.367 |

| | Mann- | | 2 Tailed | Effect |
|---|---|---|---|---|
| MS-7 | Whitney U | Z value | p-value | size |
| Control-1 | 39608.5 | −0.92587739 | 0.354509703 | 0.039 |
| Control-2 | 33331 | −0.06428 | 0.948745 | 0.003 |
| Control-3 | 32927.5 | −6.02524 | 1.69*10\hat{ }−9 | 0.245 |
| Control-4 | 6006 | −0.0362 | 0.971122 | 0.002 |
| Control-5 | 8326.5 | −1.07109 | 0.284127 | 0.057 |
| Control-6 | 9606.5 | −1.98343 | 0.047319 | 0.106 |
| Control-7 | 4130.5 | −1.40139 | 0.161097 | 0.077 |
| Control-8 | 4914 | −2.36209 | 0.018172 | 0.129 |

TABLE 8
Mann-Whitney p-values comparing frequencies of high Combo CS, IGH CDR3s (MS versus
controls) overlapping the canonical epitope.

| Control | | | | | | | | |
|---|---|---|---|---|---|---|---|---|
| group | MS-1 | MS-2 | MS-3 | MS-4 | MS-5 | MS-6 | MS-7 | MS-8 |
| Control-1 | 0.0015 | 0.0060 | 0.0028 | <0.0001 | <0.0001 | <0.0001 | 0.3545 | 0.0008 |
| Control-2 | 0.0064 | 0.0779 | 0.0099 | 0.0001 | <0.0001 | 0.0003 | 0.9487 | 0.0097 |
| Control-3 | <0.0001 | <0.0001 | <0.0001 | <0.0001 | <0.0001 | <0.0001 | <0.0001 | <0.0001 |
| Control-4 | 0.0482 | 0.2883 | 0.0783 | 0.0288 | 0.0032 | 0.0465 | 0.9711 | 0.0945 |
| Control-5 | 0.0104 | 0.0289 | 0.0140 | 0.0004 | <0.0001 | 0.0008 | 0.2841 | 0.0057 |
| Control-6 | 0.4588 | 0.4614 | 0.5718 | 0.7814 | 0.0965 | 0.9122 | 0.0473(a) | 0.9230 |
| Control-7 | 0.0081 | 0.0248 | 0.0117 | 0.0007 | 0.0002 | 0.0012 | 0.1611 | 0.0043 |
| Control-8 | 0.0008 | 0.0012 | 0.0016 | <0.0001 | <0.0001 | <0.0001 | 0.0182 | 0.0002 |

Bold data represents the standard for statistical significance. For example, in one case Control-6 has a greater frequency than MS-7.

TABLE 9
Proportion analysis comparing cumulative MS sample
high Combo CS IGH CDR3s to cumulative control high
Combo CS IGH CDR3 overlapping the canonical epitope.

| | | | Percentage of | |
|---|---|---|---|---|
| | | | unique IGH | p-value of |
| | Number of | Total | CDR3s with | proportion |
| | unique IGH | number of | frequency >61 | compared to |
| | CDR3s with | unique IGH | to total unique | control |
| Cumulative samples | frequency >61 | CDR3s | IGH CDR3s | proportion |
| MS overlapping | 186 | 1386 | 13.4199 | p < 0.0001 |
| canonical epitope | | | | |
| Control overlapping | 74 | 1055 | 7.0142 | |
| canonical epitope | | | | |

TABLE 10
Number of unique, high Combo CS IGH CDR3s at specific frequency ranges
for each MS and control sample overlapping the novel candidate epitope.

| | Number of | Number of | Number of | Number of | Number of |
|---|---|---|---|---|---|
| | unique IGH | unique IGH | unique IGH | unique IGH | unique IGH |
| | CDR3s with | CDR3s with | CDR3s with | CDR3s with | CDR3s with |
| Sample | frequency | frequency | frequency | frequency | frequency |
| Number | 10-60 | 61-110 | 111-160 | 161-210 | 211+ |
| MS-1 | 45 | 4 | 5 | 3 | 3 |
| MS-2 | 142 | 11 | 3 | 5 | 5 |
| MS-3 | 28 | 2 | 2 | 1 | 6 |
| MS-4 | 148 | 12 | 4 | 3 | 2 |
| MS-5 | 72 | 12 | 12 | 3 | 11 |
| MS-6 | 259 | 18 | 2 | 1 | 2 |
| MS-7 | 219 | 8 | 0 | 2 | 3 |
| MS-8 | 64 | 9 | 5 | 0 | 1 |
| Control-1 | 187 | 7 | 1 | 0 | 1 |
| Control-2 | 179 | 10 | 6 | 5 | 3 |
| Control-3 | 227 | 3 | 1 | 1 | 2 |
| Control-4 | 24 | 1 | 0 | 0 | 0 |
| Control-5 | 36 | 2 | 1 | 1 | 0 |
| Control-6 | 32 | 0 | 0 | 0 | 0 |
| Control-7 | 32 | 2 | 0 | 0 | 0 |
| Control-8 | 35 | 2 | 0 | 0 | 4 |

TABLE 11
Mann-Whitney p-values comparing frequencies of high Combo CS IGH CDR3s MS versus controls
overlapping the novel candidate epitope.

| Control | | | | | | | | |
|---|---|---|---|---|---|---|---|---|
| Group | MS-1 | MS-2 | MS-3 | MS-4 | MS-5 | MS-6 | MS-7 | MS-8 |
| Control- | 0.0007 | <0.0001 | 0.0017 | <0.0001 | <0.0001 | <0.0001 | <0.0001 | 0.0016 |
| 1 | | | | | | | | |
| Control- | 0.0292 | 0.0282 | 0.0247 | 0.0002 | 0.0002 | 0.0005 | 0.1034 | 0.0936 |
| 2 | | | | | | | | |
| Control- | <0.0001 | <0.0001 | <0.0001 | <0.0001 | <0.0001 | <0.0001 | <0.0001 | <0.0001 |
| 3 | | | | | | | | |
| Control- | 0.1057 | 0.3024 | 0.0580 | 0.0438 | 0.0370 | 0.0704 | 0.4883 | 0.2880 |
| 4 | | | | | | | | |
| Control- | 0.0269 | 0.0267 | 0.0212 | 0.0022 | 0.0023 | 0.0052 | 0.0536 | 0.0564 |
| 5 | | | | | | | | |
| Control- | 0.0153 | 0.0129 | 0.0182 | 0.0004 | 0.0018 | 0.0008 | 0.0197 | 0.0327 |
| 6 | | | | | | | | |
| Control- | 0.0084 | 0.0066 | 0.0080 | 0.0005 | 0.0006 | 0.0013 | 0.0130 | 0.0155 |
| 7 | | | | | | | | |
| Control- | 0.5018 | 0.7696 | 0.3327 | 0.2275 | 0.1399 | 0.3875 | 0.9051 | 0.7903 |
| 8 | | | | | | | | |

TABLE 12
Proportion analysis comparing cumulative MS sample high
Combo CS IGH CDR3s to cumulative control high Combo
CS IGH CDR3s overlapping the novel candidate epitope.

| | | | Proportion % | |
|---|---|---|---|---|
| | | | of unique | p-value of |
| | Number of | Total | IGH CDR3s with | proportion |
| | unique IGH | number of | frequency >61 | compared to |
| Cumulative | CDR3s with | unique IGH | to total unique | control |
| Samples | frequency >61 | CDR3s | IGH CDR3s | proportion |
| MS overlapping | 160 | 1137 | 14.0721 | p < 0.0001 |
| novel candidate | | | | |
| epitope | | | | |
| Control overlapping | 53 | 805 | 6.5839 | |
| novel candidate | | | | |
| epitope | | | | |

TABLE 13
Counting of individual residues within the canonical epitope for each instance of IGH CDR3
complementarity within MS samples 1-8.

| | | | | | | | | | Avg. | MS | |
|---|---|---|---|---|---|---|---|---|---|---|---|
| | | | | | | | | | by AA | Sample | Control |
| | Sample 1 | Sample 2 | Sample 3 | Sample 4 | Sample 5 | Sample 6 | Sample 7 | Sample 8 | residue | Avg | Avg |
| D | 4371 | 5648 | 20219 | 4490 | 5053 | 6636 | 5380 | 2238 | 6754.375 | 6754.375 | 2820.875 |
| E | 4371 | 5648 | 20219 | 4519 | 5088 | 6636 | 5380 | 2238 | 6762.375 | 6762.375 | 2830.75 |
| N | 4371 | 6893 | 20282 | 4732 | 5393 | 6776 | 5494 | 2238 | 7022.375 | 7022.375 | 2844.625 |
| P | 4371 | 7228 | 20293 | 4882 | 5718 | 6970 | 5583 | 2830 | 7234.375 | 7234.375 | 2814.5 |
| V | 4608 | 7561 | 20419 | 5172 | 6521 | 8273 | 6025 | 10357 | 8617 | 8617 | 3219.75 |
| V | 4642 | 8350 | 20539 | 5953 | 7796 | 8822 | 6312 | 10821 | 9154.375 | 9154.375 | 3593.5 |
| H | 4693 | 8623 | 20570 | 6023 | 7876 | 8980 | 6560 | 10880 | 9275.625 | 9275.625 | 3672.625 |
| F | 4693 | 8704 | 20570 | 6057 | 7812 | 9072 | 6539 | 10867 | 9301.75 | 9301.75 | 3683.25 |
| F | 4693 | 8753 | 20570 | 6009 | 7888 | 9072 | 6539 | 10867 | 9298.875 | 9298.875 | 3682.125 |
| K | 4693 | 8691 | 20451 | 6001 | 7888 | 9014 | 6553 | 10867 | 9269.75 | 9269.75 | 3697.5 |
| N | 4693 | 8687 | 21189 | 5983 | 7860 | 9019 | 6559 | 10867 | 9357.125 | 9357.125 | 3817.375 |
| I | 4816 | 8780 | 21202 | 6055 | 8086 | 8067 | 6628 | 11054 | 9461 | 9461 | 3925.25 |
| V | 4826 | 8918 | 21202 | 6015 | 8099 | 9034 | 6500 | 11101 | 9461.875 | 9461.875 | 3810.75 |
| T | 3889 | 6329 | 20012 | 4851 | 6389 | 6555 | 3620 | 10466 | 7763.875 | 7763.875 | 3226.125 |
| P | 3889 | 6148 | 19836 | 4808 | 6034 | 6383 | 3566 | 10466 | 7641.25 | 7641.25 | 3183.875 |
| R | 3867 | 6113 | 18676 | 4666 | 6017 | 6185 | 3515 | 10446 | 7435.625 | 7435.625 | 3159.75 |
| T | 3857 | 5996 | 18185 | 4393 | 6092 | 5780 | 3433 | 10251 | 7248.375 | 7248.375 | 2618.875 |
| P | 3857 | 5862 | 18185 | 4257 | 5893 | 5390 | 3312 | 9962 | 7089.75 | 7089.75 | 2516.625 |
| P | 1623 | 4820 | 17621 | 3744 | 5725 | 4121 | 2750 | 9750 | 6269.25 | 6269.25 | 2257.125 |
| P | 795 | 1593 | 1941 | 960 | 2766 | 1413 | 1316 | 8194 | 2372.25 | 2372.25 | 1029.5 |
| S | 889 | 1494 | 1888 | 1825 | 2076 | 1285 | 1758 | 598 | 1476.625 | 1476.625 | 1118.75 |
| Q | 1012 | 1667 | 1888 | 1855 | 2111 | 1396 | 1764 | 564 | 1532.125 | 1532.125 | 1109.25 |
| G | 941 | 1410 | 1875 | 1879 | 2070 | 1460 | 1751 | 600 | 1498.25 | 1498.25 | 1077.125 |
| K | 941 | 1410 | 1875 | 1926 | 2070 | 1460 | 1764 | 584 | 1503.75 | 1503.75 | 1077.125 |

TABLE 14
Counting of individual residues within the canonical epitope for each instance of IGH CDR3 complementarity
within Control samples 1-8.

| | Control | Control | Control | Control | Control | Control | Control | Control | Avg. by |
|---|---|---|---|---|---|---|---|---|---|
| | 1 | 2 | 3 | 4 | 5 | 6 | 7 | 8 | AA residue |
| D | 6225 | 4836 | 3894 | 774 | 4172 | 1787 | 320 | 559 | 2820.875 |
| E | 6247 | 4893 | 3894 | 774 | 4172 | 1787 | 320 | 559 | 2830.75 |
| N | 6322 | 4893 | 3808 | 796 | 4172 | 1787 | 320 | 559 | 2844.625 |
| P | 6386 | 5065 | 4098 | 817 | 4240 | 1819 | 320 | 571 | 2814.5 |
| V | 6790 | 5273 | 4843 | 1080 | 4284 | 2234 | 572 | 682 | 3219.75 |
| V | 8335 | 5779 | 5460 | 1080 | 4391 | 2296 | 671 | 736 | 3593.5 |
| H | 8465 | 6017 | 5617 | 1080 | 4405 | 2390 | 671 | 736 | 3672.625 |
| F | 8475 | 6107 | 5592 | 1080 | 4415 | 2390 | 671 | 736 | 3683.25 |
| F | 8475 | 6135 | 5569 | 1080 | 4415 | 2390 | 671 | 722 | 3682.125 |
| K | 8635 | 6108 | 5538 | 1101 | 4415 | 2390 | 671 | 722 | 3697.5 |
| N | 9625 | 6170 | 5516 | 1040 | 4415 | 2390 | 671 | 712 | 3817.375 |
| I | 9838 | 6273 | 6043 | 1040 | 4425 | 2400 | 671 | 712 | 3925.25 |
| V | 9809 | 6091 | 6163 | 1040 | 4400 | 2400 | 671 | 712 | 3810.75 |
| T | 7476 | 5030 | 4767 | 955 | 4268 | 2191 | 507 | 615 | 3226.125 |
| P | 7277 | 5030 | 4767 | 858 | 4268 | 2191 | 507 | 573 | 3183.875 |
| R | 7231 | 4984 | 4695 | 858 | 4268 | 2173 | 496 | 573 | 3159.75 |
| T | 7007 | 4907 | 4298 | 597 | 986 | 2142 | 455 | 559 | 2618.875 |
| P | 6960 | 4877 | 3929 | 583 | 933 | 1885 | 434 | 532 | 2516.625 |
| P | 6461 | 4025 | 3796 | 499 | 883 | 1634 | 296 | 463 | 2257.125 |
| P | 3446 | 1941 | 2093 | 106 | 228 | 218 | 141 | 63 | 1029.5 |
| S | 3976 | 1924 | 2129 | 123 | 182 | 367 | 141 | 108 | 1118.75 |
| Q | 3959 | 1932 | 2063 | 123 | 179 | 367 | 129 | 122 | 1109.25 |
| G | 4032 | 1585 | 2080 | 123 | 179 | 367 | 129 | 122 | 1077.125 |
| K | 4043 | 1584 | 2070 | 123 | 179 | 367 | 129 | 122 | 1077.125 |

TABLE 15
Counting of individual residues within the candidate epitope for each instance of IGH CDR3
complementarity within MS samples 1-8.

| | | | | | | | | | Avg. | MS | |
|---|---|---|---|---|---|---|---|---|---|---|---|
| | | | | | | | | | by AA | Sample | Control |
| | Sample 1 | Sample 2 | Sample 3 | Sample 4 | Sample 5 | Sample 6 | Sample 7 | Sample 8 | residue | Avg | Avg |
| A | 4832 | 5827 | 2168 | 5098 | 8402 | 7648 | 4284 | 2697 | 5119.5 | 5119.5 | 2355.75 |
| D | 4832 | 5861 | 2179 | 5132 | 8596 | 7809 | 5741 | 2697 | 5355.875 | 5355.875 | 2412.625 |
| P | 4832 | 5801 | 2179 | 5132 | 8664 | 8002 | 5833 | 2697 | 5392.5 | 5392.5 | 2434.875 |
| G | 4832 | 5812 | 2179 | 5132 | 8679 | 8002 | 5833 | 2275 | 5343 | 5343 | 2437.25 |
| S | 4832 | 5812 | 2179 | 5196 | 8679 | 8002 | 5833 | 2275 | 5351 | 5351 | 2439.25 |
| R | 4832 | 5812 | 2179 | 5208 | 8679 | 8025 | 5793 | 2275 | 5350.375 | 5350.375 | 2442.5 |
| P | 4842 | 6549 | 3229 | 5243 | 8704 | 8025 | 5819 | 2275 | 5585.75 | 5585.75 | 2449 |
| H | 4895 | 6600 | 3241 | 5284 | 8754 | 8053 | 6029 | 2275 | 5641.375 | 5641.375 | 2480.875 |
| L | 4921 | 6752 | 3241 | 5392 | 8754 | 8053 | 6045 | 2275 | 5679.125 | 5679.125 | 2493.625 |
| I | 4897 | 6741 | 3231 | 5435 | 8826 | 8014 | 6103 | 2275 | 5690.25 | 5690.25 | 2497.125 |
| R | 4897 | 6741 | 3231 | 5435 | 8848 | 8052 | 6174 | 2275 | 5706.625 | 5706.625 | 2499.75 |
| L | 4897 | 7572 | 3231 | 5525 | 9833 | 8172 | 6357 | 2296 | 5985.375 | 5985.375 | 2524.5 |
| F | 4897 | 7542 | 3231 | 5511 | 9833 | 8153 | 6357 | 2296 | 5977.5 | 5977.5 | 2523.125 |
| S | 4775 | 7175 | 3180 | 4548 | 9375 | 7695 | 6193 | 2008 | 5618.625 | 5618.625 | 2437.25 |
| R | 1555 | 3317 | 1321 | 1381 | 2536 | 2758 | 1526 | 791 | 1898.125 | 1898.125 | 1097.125 |
| D | 801 | 3330 | 1203 | 1109 | 2375 | 1937 | 1310 | 652 | 1589.625 | 1589.625 | 1034.625 |
| A | 756 | 3323 | 1217 | 1061 | 2355 | 1844 | 1192 | 634 | 1547.75 | 1547.75 | 988.125 |
| P | 177 | 2724 | 1128 | 742 | 1535 | 1329 | 996 | 430 | 1132.625 | 1132.625 | 523.625 |
| G | 200 | 2127 | 1076 | 581 | 1410 | 753 | 699 | 80 | 865.75 | 865.75 | 192.25 |
| R | 200 | 2139 | 1122 | 617 | 1342 | 677 | 769 | 136 | 875.25 | 875.25 | 206.625 |
| E | 200 | 2128 | 1122 | 595 | 1345 | 677 | 714 | 136 | 864.625 | 864.625 | 208.125 |
| D | 200 | 2128 | 338 | 595 | 1308 | 681 | 635 | 175 | 757.5 | 757.5 | 196 |
| N | 142 | 2070 | 60 | 524 | 1161 | 627 | 612 | 281 | 684.625 | 684.625 | 170.5 |
| T | 132 | 1163 | 60 | 422 | 1161 | 279 | 549 | 245 | 501.375 | 501.375 | 140.125 |

TABLE 16
Counting of individual residues within the candidate epitope for each instance of IGH CDR3 complementarity
within Control samples 1-8.

| | Control | Control | Control | Control | Control | Control | Control | Control | Avg. by |
|---|---|---|---|---|---|---|---|---|---|
| | 1 | 2 | 3 | 4 | 5 | 6 | 7 | 8 | AA residue |
| A | 3794 | 5520 | 3596 | 474 | 804 | 433 | 712 | 3513 | 2355.75 |
| D | 4094 | 5612 | 3598 | 474 | 865 | 433 | 712 | 3513 | 2412.625 |
| P | 4094 | 5656 | 3637 | 474 | 922 | 433 | 736 | 3527 | 2434.875 |
| G | 4094 | 5675 | 3637 | 474 | 922 | 433 | 736 | 3527 | 2437.25 |
| S | 4094 | 5675 | 3653 | 474 | 922 | 433 | 736 | 3527 | 2439.25 |
| R | 4110 | 5675 | 3663 | 474 | 922 | 433 | 736 | 3527 | 2442.5 |
| P | 4120 | 5691 | 3679 | 474 | 932 | 433 | 736 | 3527 | 2449 |
| H | 4215 | 5705 | 3775 | 474 | 972 | 443 | 736 | 3527 | 2480.875 |
| L | 4268 | 5729 | 3789 | 474 | 983 | 443 | 736 | 3527 | 2493.625 |
| I | 4284 | 5726 | 3778 | 500 | 983 | 443 | 736 | 3527 | 2497.125 |
| R | 4284 | 5726 | 3799 | 500 | 983 | 443 | 736 | 3527 | 2499.75 |
| L | 4369 | 5785 | 3853 | 500 | 983 | 443 | 736 | 3527 | 2524.5 |
| F | 4358 | 5785 | 3853 | 500 | 983 | 443 | 736 | 3527 | 2523.125 |
| S | 4191 | 5630 | 3596 | 472 | 955 | 414 | 723 | 3517 | 2437.25 |
| R | 1804 | 2364 | 1033 | 155 | 421 | 149 | 83 | 2668 | 1097.125 |
| D | 1753 | 2203 | 936 | 98 | 421 | 129 | 83 | 2654 | 1034.625 |
| A | 1662 | 2099 | 928 | 98 | 421 | 45 | 83 | 2569 | 988.125 |
| P | 1166 | 1660 | 676 | 76 | 421 | 45 | 23 | 122 | 523.625 |
| G | 380 | 525 | 423 | 46 | 129 | 35 | 0 | 0 | 192.25 |
| R | 380 | 545 | 421 | 59 | 195 | 35 | 0 | 18 | 206.625 |
| E | 380 | 562 | 431 | 59 | 195 | 20 | 0 | 18 | 208.125 |
| D | 380 | 495 | 411 | 59 | 195 | 10 | 0 | 18 | 196 |
| N | 304 | 373 | 455 | 60 | 144 | 10 | 0 | 18 | 170.5 |
| T | 271 | 205 | 433 | 40 | 144 | 433 | 0 | 18 | 140.125 |

TABLE 17
Twenty examples of IGH CDR3-antigen amino acid (AA) alignment and residue frequency
counts utilizing IGH CDR3s from sample MS-8 and MBP isoform 5 (as the antigen).

| | | | | IGH CDR3- |
|---|---|---|---|---|
| | | IGH CDR3-MBP | | MBP AA |
| | | AA alignment | | residue |
| | | residue range | | frequency |
| | IGH CDR3-MBP AA alignment | from Adaptive | Combo | count from |
| IGH CDR3 | from Adaptive Match | Match utput | CS | iReceptor |
| CAADGYSYGPRHNAFDIW | FLPRHRDTGILDSIGRFF | 28-46 | 8.71 | 25 |
| (SEQ ID NO: 11) | (SEQ ID NO: 31) | | | |
| CAAGTRSSGGSCYSLGYW | GFKGVDAQGTLSKIFKLG | 140-158 | 7.66 | 28 |
| (SEQ ID NO: 12) | (SEQ ID NO: 32) | | | |
| CAAGYYYDSSGYDFQHW | PVVHFFKNIVTPRTPPP | 85-102 | 6.77 | 18 |
| (SEQ ID NO: 13) | (SEQ ID NO: 33) | | | |
| CAAIAAAGLAVW | PVVHFFKNIVTP | 85-97 | 6.63 | 18 |
| (SEQ ID NO: 14) | (SEQ ID NO: 34) | | | |
| CAEDVGGYWVHQLGYW | LPRHRDTGILDSIGRF | 29-45 | 7.42 | 54 |
| (SEQ ID NO: 15) | (SEQ ID NO: 35) | | | |
| CAEEGGSGWPYFDYW | GFKGVDAQGTLSKIF | 140-155 | 7.06 | 122 |
| (SEQ ID NO: 16) | (SEQ ID NO: 36) | | | |
| CAEGRFGPYSSGWYASW | FKGVDAQGTLSKIFKLG | 141-158 | 7.53 | 21 |
| (SEQ ID NO: 17) | (SEQ ID NO: 37) | | | |
| CAGATVIPYNWFDPW | GVDAQGTLSKIFKLG | 143-158 | 7.19 | 13 |
| (SEQ ID NO: 18) | (SEQ ID NO: 38) | | | |
| CAGCPGGSSWYYYFDYW | FKGVDAQGTLSKIFKLG | 141-158 | 7.21 | 28 |
| (SEQ ID NO: 19) | (SEQ ID NO: 39) | | | |
| CAGDPPYCSNGVCSGPYYNGLDVW | YKSAHKGFKGVDAQGTLSKIFKLG | 134-158 | 9.39 | 17 |
| (SEQ ID NO: 20) | (SEQ ID NO: 40) | | | |
| CAGELIAVAGPIDYW | GFKGVDAQGTLSKIF | 140-155 | 8.21 | 11 |
| (SEQ ID NO: 21) | (SEQ ID NO: 41) | | | |
| CAGRSSTAYYYIMDIW | KGVDAQGTLSKIFKLG | 142-158 | 8.85 | 15 |
| (SEQ ID NO: 22) | (SEQ ID NO: 42) | | | |
| CAGRSSTAYYYTMDIW | KGVDAQGTLSKIFKLG | 142-158 | 8.25 | 516 |
| (SEQ ID NO: 23) | (SEQ ID NO: 42) | | | |
| CAGVSYYYDSSGYYYEPFDYW | TGILDSIGRFFGGDRGAPKRG | 35-56 | 9.07 | 23 |
| (SEQ ID NO: 24) | (SEQ ID NO: 43) | | | |
| CAHGKLAGPFDSW | FKGVDAQGTLSKI | 141-154 | 6.81 | 143 |
| (SEQ ID NO: 25) | (SEQ ID NO: 44) | | | |
| CAHGRYLDGAIDYW | VDAQGTLSKIFKLG | 144-158 | 7.63 | 629 |
| (SEQ ID NO: 26) | (SEQ ID NO: 45) | | | |
| CAHKKLFGELPDYW | VDAQGTLSKIFKLG | 144-158 | 7.91 | 99 |
| (SEQ ID NO: 27) | (SEQ ID NO: 45) | | | |
| CAHLTITFGGTPRDDAFDSW | GILDSIGRFFGGDRGAPKRG | 36-56 | 10.67 | 34 |
| (SEQ ID NO: 28) | (SEQ ID NO: 46) | | | |
| CAHRLGPLANRAAYFDYW | ILDSIGRFFGGDRGAPKR | 37-55 | 8.16 | 67 |
| (SEQ ID NO: 29) | (SEQ ID NO: 47) | | | |
| CAHRQGYSYGIADYW | GVDAQGTLSKIFKLG | 143-158 | 6.95 | 36 |
| (SEQ ID NO: 30) | (SEQ ID NO: 48) | | | |

Note,
the represented IGH CDR3s are an example taken after the removal of IGH CDR3s with a Combo CS less than 6.0 or a frequency less than 10. Additionally, the residue range below is from the Adaptive Match output, which does not align exactly with the Uniport residue numbers. Specifically, the residue numbers indicated below are one less for each AA residue than would be indicated by Uniport and one less than is indicated in all other text in this report. Combo CSs were rounded to the nearest hundredth.

TABLE 18
Unique Residue Ratios (URR) for the AA residues 10-40 of
MBP Isoform 5, used as the antigen, and using IGH CDR3s
from immune repertoire sample MS-8 (from iReceptor.org).

| | | | URR |
|---|---|---|---|
| | IGH CDR3-antigen | | (This URR value prevents any one highly |
| | AA residue | | frequent IGH CDR3 from immune |
| | frequency count, | | repertoire file leading to a bias in the |
| | established by | Total Number | interpretation of the overlaps of IGH |
| | the frequency of | of Unique | CDR3s and the given AA residues. That |
| | the IGH CDR3s | IGH CDR3s | is, if almost all CDR3s that overlap the |
| | in the original | that overlap | residue are highly amplified in the |
| Residue | immune repertoire | the AA | immune repertoire results, the URR value |
| Number | dataset | residue | is higher.) |
| 10 | 46 | 2 | 23.00 |
| 11 | 46 | 2 | 23.00 |
| 12 | 46 | 2 | 23.00 |
| 13 | 46 | 2 | 23.00 |
| 14 | 84 | 4 | 21.00 |
| 15 | 96 | 5 | 19.20 |
| 16 | 96 | 5 | 19.20 |
| 17 | 123 | 6 | 20.50 |
| 18 | 133 | 7 | 19.00 |
| 19 | 178 | 9 | 19.78 |
| 20 | 279 | 13 | 21.46 |
| 21 | 382 | 16 | 23.88 |
| 22 | 472 | 17 | 27.76 |
| 23 | 472 | 17 | 27.76 |
| 24 | 553 | 18 | 30.72 |
| 25 | 624 | 20 | 31.20 |
| 26 | 1050 | 26 | 40.38 |
| 27 | 3471 | 35 | 99.17 |
| 28 | 6075 | 73 | 83.22 |
| 29 | 6587 | 78 | 84.45 |
| 30 | 6850 | 86 | 79.65 |
| 31 | 7126 | 87 | 81.91 |
| 32 | 8939 | 107 | 83.54 |
| 33 | 10135 | 124 | 81.73 |
| 34 | 10424 | 131 | 79.57 |
| 35 | 12161 | 145 | 83.87 |
| 36 | 13051 | 168 | 77.68 |
| 37 | 23918 | 292 | 81.91 |
| 38 | 25406 | 300 | 84.69 |
| 39 | 25423 | 301 | 84.46 |
| 40 | 26519 | 303 | 87.52 |

Note:
The alignment of each IGH CDR3 to a particular AA residue on the antigen is carried out by adaptivematch.com. Once the alignment for each IGH CDR3 is determined, the frequency of each IGH CDR3 from an immune repertoire file overlapping with a specific antigen AA residue is summed. This produces the IGH CDR3-antigen AA residue frequency count for each AA residue. Rounded to the nearest hundredth.

TABLE 19
Weighted Unique Residue Ratio (WURR) Calculation
for Residue 87 of MBP isoform 5 (as an example).

| | URR for | Sample | | |
|---|---|---|---|---|
| Sample | Residue 87 | Size* | Weight | WURR |
| MS-1 | 89.27 | 52416 | 0.06 | 5.72 |
| MS-2 | 48.55 | 106407 | 0.13 | 6.31 |
| MS-3 | 270.25 | 112066 | 0.14 | 37.00 |
| MS-4 | 39.16 | 95907 | 0.12 | 4.59 |
| MS-5 | 77.19 | 131305 | 0.16 | 12.38 |
| MS-6 | 33.93 | 129842 | 0.16 | 5.38 |
| MS-7 | 26.41 | 95546 | 0.12 | 3.08 |
| MS-8 | 133.59 | 94947 | 0.12 | 15.50 |
| Total | | 818436 | 1 | 89.97 |

This calculation provides IGH CDR3-MBP AA residue value (in this case, representing the beginning of the canonical epitope) that takes into consideration all immune repertoire samples of a given study.
Note:
All values (except sample size) were rounded to the nearest hundredth; also note that the sample size reflects maintaining only the IGH CDR3s that were repeated 10 or more times in the original immune repertoire sample.

TABLE 20
WURR approach to identifying candidate IGH epitopes, using MS versus COVID versus

| | | | | |
|---|---|---|---|---|
| | | | Average WURR | |
| | | Average WURR | difference | |
| | | difference of | outside of the | |
| | | potential | range of the | |
| | | epitope | potential | |
| Range | Sequence | candidate | epitope candidate | p value |

COVID controls

| | | | | |
|---|---|---|---|---|
| 87-102 | VVHFFKNIVTPRTPPP | 58.65 | 29.49 | <0.0001 |
| | (SEQ ID NO: 7) | | | |
| 118-130 | GAEGQRPGFGYGG | 65.48 | 29.48 | <0.0001 |
| | (SEQ ID NO: 5) | | | |

Healthy controls

| | | | | |
|---|---|---|---|---|
| 87-102 | VVHFFKNIVTPRTPPP | 50.93 | 20.9 | <0.0001 |
| | (SEQ ID NO: 7) | | | |
| 118-131 | GAEGQRPGFGYGGR | 64.37 | 20.13 | <0.0001 |
| | (SEQ ID NO: 50) | | | |

Note, the AA sequences indicated here were originally established by a high difference in WURR values, in comparison to the same region where the WURR values were based on COVID IGH CDR3s. After that process, the differences in WURR values over the indicated regions were compared, by T-test, to the difference in WURR values for the remainder of MBP isoform 5.
a, Note, the following two AA set was also yield by the indicated WURR approach, however, is informally discounted due to the small size. The data for this sequence is as follows, 19-20, AS, 52.27, 31.85, <0.0001. healthy control IGH CDR3 datasets and the MBP isoform 5 amino acid sequence.

TABLE 21
Examples of Combo CS matching for WURR-Combo CS approach.

| | |
|---|---|
| Candidate Epitope Sequence | VVHFFKNIVTPRTPPP |
| | (SEQ ID NO: 7) |

Exact Match

| | |
|---|---|
| IGH CDR3 Sequence | CARVLDWRAGSPTSPW |
| | (SEQ ID NO: 51) |
| Sequence where IGH CDR3 | VVHFFKNIVTPRTPPP |
| Compliments | (SEQ ID NO: 7) |

Internal Match

| | |
|---|---|
| IGH CDR3 Sequence | CARRMTVVAEYNFWSSYSSGPSWFDPW |
| | (SEQ ID NO: 52) |
| Sequence where IGH CDR3 | QDENPVVHFFKNIVTPRTPPPSQGKGR |
| Compliments | (SEQ ID NO: 53) |

Partial Overlap (Not Included)

| | |
|---|---|
| IGH CDR3 Sequence | CAKTRPHLVLVTVPVW |
| | (SEQ ID NO: 54) |
| Sequence where IGH CDR3 | TQDENPVVHFFKNIVT |
| Compliments | (SEQ ID NO: 55) |

TABLE 22
WURR-Combo CS approach with MS versus COVID controls for MBP isoform 5.

| | | | | | |
|---|---|---|---|---|---|
| | | Average MS | Average MS | | |
| | | Combo CSs | Combo CSs | | |
| | | when high | represented | | |
| | | difference | by the | | |
| | | WURR AA | high difference | | IEDB |
| | | sequence is | WURR AA | | Partial |
| Range | Sequence | not included | sequence | p value | Matches |

COVID controls

| | | | | | |
|---|---|---|---|---|---|
| 87-102 | VVHFFKNIVTPRTPP | 8.3 | 10.4 | <0.0001 | 16 |
| | P (SEQ ID NO: 7) | | | | |
| 118-130 | GAEGQRPGFGYGG | 8.3 | 8.71 | 0.0026 | 5 |
| | (SEQ ID NO: 5) | | | | |

Healthy controls

| | | | | | |
|---|---|---|---|---|---|
| 87-102 | VVHFFKNIVTPRTPP | 8.3 | 10.4 | <0.0001 | 16 |
| | P (SEQ ID NO: 7) | | | | |
| 118-131 | GAEGQRPGFGYGGR | 8.3 | 8.73 | 0.0015 | 5 |
| | (SEQ ID NO: 50) | | | | |

Note,
the candidate AA sequences indicated here were originally established by high difference in WURR values, using the COVID IGH CDR3s as the control. After that process, the Combo CSs of IGH CDR3s that internally or exactly matched candidate AA sequences were compared, by T-test, to the Combo CSs for the IGH CDR3s that did not internally or exactly match for the remainder of MBP isoform 5. For the IEDB-epitope match protocol, see Methods.

TABLE 23
WURR approach to identifying candidate IGH epitopes for various antigenic sequences.

| | | | | | Average | |
|---|---|---|---|---|---|---|
| | | Range | | | WURR | |
| | | of AA | | Average | difference | |
| | | in the | | WURR | outside of | |
| | | poly- | | difference | the range | Hetero- |
| | MS/CD | peptide | Sequence of high | for the | of the | scedastic |
| | patient | being | difference WURR | candidate | candidate | T-test |
| Protein | CDR3s | analyzed | (candidate epitope) | epitope | epitope | p-value |
| Proteolipid | COVID controls | | | | | |
| Protein | MS | 81-84 | LYGA | 88.38 | 25.5 | 0.0062 |
| | | | (SEQ ID NO: 49) | | | |
| | | 115-117 | ATV | 230.07 | 24.2 | <0.0001 |
| | Healthy controls | | | | | |
| | MS | 81-84 | LYGA | 70.49 | 16.89 | 0.0086 |
| | | | (SEQ ID NO: 49) | | | |
| | | 115-117 | ATV | 240.18 | 15.26 | <0.0001 |
| Alpha/beta- | Healthy controls^{a} | | | | | |
| gliadin | CD | 3-29 | TFLILALLAIVATTARIA VR | 278.72 | 92.38 | <0.0001 |
| MM1 | | | VPVPQLQ (SEQ ID NO: 56) | | | |
| | | 161-177 | IPCRDVVLQQHSIAYGS | 276.86 | 98.83 | <0.0001 |
| | | | (SEQ ID NO: 57) | | | |
| | | 200-217 | IPEQSRCQAIHNVVHAII | 259.59 | 100.03 | <0.0001 |
| | | | (SEQ ID NO: 58) | | | |
| | | 270-291 | LPQFEEIRNLALETLPAMC | 244.95 | 98.18 | <0.0001 |
| | | | NVY | | | |
| | | | (SEQ ID NO: 8) | | | |
| Prolamin | Healthy controls | | | | | |
| | CD | 106-134 | QILQQILQQQLIPCRDVVLQ | 266.86 | 84.91 | <0.0001 |
| | | | QPNIAHASS | | | |
| | | | (SEQ ID NO: 59) | | | |
| | | 164-176 | IHNVIHAIILHHQ | 253.10 | 96.27 | <0.0001 |
| | | | (SEQ ID NO: 60) | | | |
| | | 230-279 | NPQAQGFVQPQQLPQFEEI | 276.55 | 66.59 | <0.0001 |
| | | | RNLALQTLPAMCNVYIPPY | | | |
| | | | CSTTIAPFGIFS | | | |
| | | | (SEQ ID NO: 61) | | | |
| Gamma | Healthy controls^{b} | | | | | |
| Gliadin | CD | 3-24 | TLLILTILAMAITIGTANIQV | 258.46 | 97.79 | <0.0001 |
| | | | D | | | |
| | | | (SEQ ID NO: 62) | | | |
| | | 253-273 | QGIDIFLPLSQHEQVGQGSL | 269.51 | 97.56 | <0.0001 |
| | | | V | | | |
| | | | (SEQ ID NO: 63) | | | |
| | | 277-328 | GIIQPQQPAQLEAIRSLVLQ | 260.38 | 72.30 | <0.0001 |
| | | | TLPSMCNVYVPPECSIMRA | | | |
| | | | PFASIVAGIGGQ | | | |
| | | | (SEQ ID NO: 64) | | | |
| | COVID controls^{c} | | | | | |
| EBV Nuclear | MS | 66-86 | HRDGVRRPQKRPSCIGCKG | 69.85 | 12.03 | <0.0001 |
| Antigen 1 | | | TH | | | |
| | | | (SEQ ID NO: 65) | | | |
| | | 518-522 | YNLRR (SEQ ID NO: 66) | 36.80 | 13.73 | <0.0001 |
| | | 602-624 | DGVDLPPWFPPMVEGAAA | 62.52 | 12.11 | <0.0001 |
| | | | EGDDG | | | |
| | | | (SEQ ID NO: 67) | | | |
| | Healthy controlsd | | | | | |
| | MS | 35-52 | GGDNHGRGRGRGRGRGG | 38.01 | 2.38 | <0.0001 |
| | | | G | | | |
| | | | (SEQ ID NO: 68) | | | |
| | | 66-86 | HRDGVRRPQKRPSCIGCKG | 45.24 | 1.97 | <0.0001 |
| | | | TH | | | |
| | | | (SEQ ID NO: 65) | | | |
| | | 500-508 | EGTWVAGVF | 25.56 | 3.06 | <0.0001 |
| | | | (SEQ ID NO: 69) | | | |
| | | 518-541 | YNLRRGTALAIPQCRLTPL | 27.30 | 2.45 | <0.0001 |
| | | | SRLPF | | | |
| | | | (SEQ ID NO: 70) | | | |
| | | 552-562 | GPLRESIVCYF | 27.12 | 2.96 | <0.0001 |
| | | | (SEQ ID NO: 71) | | | |
| | | 564-569 | VFLQTH (SEQ ID NO: 72) | 28.02 | 3.14 | <0.0001 |
| | | 576-595 | KDAIKDLVMTKPAPTCNIR | 28.45 | 2.57 | <0.0001 |
| | | | V | | | |
| | | | (SEQ ID NO: 73) | | | |
| | | 610-624 | FPPMVEGAAAEGDDG | 56.64 | 2.11 | <0.0001 |
| | | | (SEQ ID NO: 74) | | | |

Note, the following two AA sets were also yield by the indicated WURR approach, however, are
informally discounted due to the small size. The data for these sequences are as follows:

| | | | | | |
|---|---|---|---|---|---|
| a | 306-308 | TN | 285.14 | 106.84 | 0.01944 |
| b | 30-31 | QW | 234.04 | 107.64 | <0.0001 |
| c | 507-508 | VF | 36.03 | 13.84 | <0.0001 |
| | 552-553 | GP | 45.65 | 13.81 | 0.0022 |
| d | 607-608 | PP | 24.57 | 3.31 | <0.0001 |

Note,
the AA sequences indicated here were originally established by a high difference in WURR values. Then, the differences in high difference WURR values versus the WURR values represented by the remainder of the polypeptide were compared, by T-test. Also note that the remainder of the polypeptide, used for comparison to a given, single, continuous AA sequence defined by a high difference WURR value, would also potentially contain other high difference WURR AA sequences.

TABLE 24
WURR-Combo CS approach to identifying candidate IGH epitopes for various antigenic
sequences.

| | | | | Average | Average MS | | |
|---|---|---|---|---|---|---|---|
| | | | | MS Combo | Combo CSs | | |
| | | | | CSs when high | represented | | |
| | | | | difference | by the high | | |
| | MS/CD | | | WURR AA | difference | | IEDB |
| Antigenic | Patient | | | sequence is | WURR AA | p | Partial |
| Sequence | CDR3s | Range | Sequence | not included | sequence | value | Matches |
| Proteolipid | COVID controls | | | | | | |
| Protein | MS | 81-84 | LYGA (SEQ ID NO: 49) | 8.89 | 9.41 | 0.0284 | 0 |
| | | 115-117 | ATV | 8.89 | 9.42 | 0.0188 | 4 |
| | Healthy controls | | | | | | |
| | MS | 81-84 | LYGA (SEQ ID NO: 49) | 8.89 | 9.4 | 0.0284 | 0 |
| | | 115-117 | ATV | 8.89 | 9.42 | 0.0188 | 4 |
| Alpha/beta- | Healthy controls | | | | | | |
| gliadin | CD | 3-29 | TFLILALLAIVATTARIAVR | 9.17 | 13.24 | <0.0001 | 13 |
| MM1 | | | VPVPQLQ | | | | |
| | | | (SEQ ID NO: 56) | | | | |
| | | 161-177 | IPCRDVVLQQHSIAYGS | 9.17 | 8.24 | | |
| | | | (SEQ ID NO: 57) | | | | |
| | | 200-217 | IPEQSRCQAIHNVVHAII | 9.17 | 9.38 | 0.0059 | 18 |
| | | | (SEQ ID NO: 58) | | | | |
| | | 270-291 | LPQFEEIRNLALETLPAMC | 9.15 | 11.75 | <0.0001 | 17 |
| | | | NVY | | | | |
| | | | (SEQ ID NO: 8) | | | | |
| Prolamin | Healthy controls | | | | | | |
| | CD | 106-134 | QILQQILQQQLIPCRDVVL | * | * | * | 22 |
| | | | QQPNIAHASS | | | | |
| | | | (SEQ ID NO: 59) | | | | |
| | | 164-176 | IHNVIHAIILHHQ | 8.31 | 7.87 | | |
| | | | (SEQ ID NO: 60) | | | | |
| | | 230-279 | NPQAQGFVQPQQLPQFEEI | * | * | * | 32 |
| | | | RNLALQTLPAMCNVYIPP | | | | |
| | | | YCSTTIAPFGIFS | | | | |
| | | | (SEQ ID NO: 61) | | | | |
| Gamma | Healthy controls | | | | | | |
| Gliadin | CD | 3-24 | TLLILTILAMAITIGTANIQ | 8.60 | 11.30 | <0.0001 | 0 |
| | | | VD | | | | |
| | | | (SEQ ID NO: 62) | | | | |
| | | 253-273 | QGIDIFLPLSQHEQVGQGS | 8.62 | 10.74 | 0.0001 | 2 |
| | | | LV | | | | |
| | | | (SEQ ID NO: 63) | | | | |
| | | 277-328 | GIIQPQQPAQLEAIRSLVLQ | * | * | * | 5 |
| | | | TLPSMCNVYVPPECSIMRA | | | | |
| | | | PFASIVAGIGGQ | | | | |
| | | | (SEQ ID NO: 64) | | | | |
| | COVID controls | | | | | | |
| EBVN | MS | 66-86 | HRDGVRRPQKRPSCIGCK | 8.76 | 10.05 | 0.2665 | |
| | | | GTH | | | | |
| | | | (SEQ ID NO: 65) | | | | |
| | | 518-522 | YNLRR (SEQ ID NO: 66) | 8.73 | 9.77 | <0.0001 | 18 |
| | | 602-624 | DGVDLPPWFPPMVEGAAA | 8.76 | 14.30 | * | 19 |
| | | | EGDDG(a) | | | | |
| | | | (SEQ ID NO: 67) | | | | |
| | Healthy controls | | | | | | |
| | MS | 35-52 | GGDNHGRGRGRGRGRGG | 8.76 | 9.69 | 0.0017 | 36 |
| | | | G (SEQ ID NO: 68) | | | | |
| | | 66-86 | HRDGVRRPQKRPSCIGCK | 8.76 | 10.05 | 0.2665 | |
| | | | GTH | | | | |
| | | | (SEQ ID NO: 65) | | | | |
| | | 500-508 | EGTWVAGVF | 8.74 | 9.36 | <0.0001 | 15 |
| | | | (SEQ ID NO: 69) | | | | |
| | | 518-541 | YNLRRGTALAIPQCRLTPL | 8.76 | 12.29 | 0.0033 | 25 |
| | | | SRLPF | | | | |
| | | | (SEQ ID NO: 70) | | | | |
| | | 552-562 | GPLRESIVCYF | 8.77 | 8.95 | 0.2905 | |
| | | | (SEQ ID NO: 71) | | | | |
| | | 564-569 | VFLQTH | 8.63 | 9.59 | <0.0001 | 7 |
| | | | (SEQ ID NO: 72) | | | | |
| | | 576-595 | KDAIKDLVMTKPAPTCNIR | 8.65 | 11.34 | <0.0001 | 22 |
| | | | V (SEQ ID NO: 73) | | | | |
| | | 610-624 | FPPMVEGAAAEGDDG | 8.76 | 11.36 | 0.0934 | |
| | | | (SEQ ID NO: 74) | | | | |

Note,
the AA sequences indicated here were originally established by a high difference in WURR values, in comparison to the same region where the WURR values were based on COVID IGH CDR3s. After that process, the differences in WURR values over the indicated regions were compared, by T-test, to the difference in WURR values for the remainder of MBP isoform 5. For the IEDB-epitope match protocol, see Methods.
a, Sequence of indeterminant significance due to only one IGH CDR3 within the samples internally aligning with the potential epitope. T-test evaluation is impossible without a sample size of 2 or greater in both groups, those being IGH CDR3s exactly or internally aligning the potential epitope and all other IGH CDR3s.

TABLE 25
Chi-square analysis of IEDB Matching Data

| | Number of candidate | Number of candidate |
|---|---|---|
| Number of unique | epitopes with at least | epitopes with no |
| candidate epitopes | 1 partial match within | partial matches |
| matched against | its antigen's | within its antigen's |
| the IEDB database | respective database | respective database |
| 20 | 18 | 2 |

| | |
|---|---|
| Difference | 80% |
| 95% CI | 51.5695% to 90.2012% |
| Chi-squared | 24.960 |
| DF | 1 |
| Significance level | p < 0.0001 |

SEQUENCES

1. SEQ ID NO: 1-MBP epitope amino acids 83 to 106
DENPVVHFFKNIVTPRTPPPSQGK
2. SEQ ID NO: 2-Candidate Antigen peptide
ADPGSRPHLIRLFSRDAPGREDNT
3. SEQ ID NO: 3-Example Antigen Peptide
TQDENPVVHF
4. SEQ ID NO: 4-Example Antigen Peptide
TQDQNPVVHF
5. SEQ ID NO: 5-Example Epitope Peptide
GAEGQRPGFGYGG
6. SEQ ID NO: 6-Example Database Entry Peptide
WGAEGQKPGFGYGG
7. SEQ ID NO: 7-MBP epitope amino acids 87 to 102 of MBP isoform 5
VVHFFKNIVTPRTPPP
8. SEQ ID NO: 8-MM1 epitope amino acids 270 to 291
LPQFEEIRNLALETLPAMCNVY
9. SEQ ID NO: 9-MM1 epitope amino acids 279 to 288 with E282Q mutation
LALQTLPAMC
10. SEQ ID NO: 10-Candidate Antigen
MASQKRPSQRHGSKYLATASTMDHARHGFLPRHRDTGILDSIGRFFGGDRGAPKRGSGKDSH
HPARTAHYGSLPQKSHGRTQDENPVVHFFKNIVTPRTPPPSQGKGRGLSLSRFSWGAEGQRPG
FGYGGRASDYKSAHKGFKGVDAQGTLSKIFKLGGRDSRSGSPMARR
11. SEQ ID NO: 11-IGH CDR3
CAADGYSYGPRHNAFDIW
12. SEQ ID NO: 12-IGH CDR3
CAAGTRSSGGSCYSLGYW
13. SEQ ID NO: 13-IGH CDR3
CAAGYYYDSSGYDFQHW
14. SEQ ID NO: 14-IGH CDR3
CAAIAAAGLAVW
15. SEQ ID NO: 15-IGH CDR3
CAEDVGGYWVHQLGYW
16. SEQ ID NO: 16-IGH CDR3
CAEEGGSGWPYFDYW
17. SEQ ID NO: 17-IGH CDR3
CAEGREGPYSSGWYASW
18. SEQ ID NO: 18-IGH CDR3
CAGATVIPYNWFDPW
19. SEQ ID NO: 19-IGH CDR3
CAGCPGGSSWYYYFDYW
20. SEQ ID NO: 20-IGH CDR3
CAGDPPYCSNGVCSGPYYNGLDVW
21. SEQ ID NO: 21-IGH CDR3
CAGELIAVAGPIDYW
22. SEQ ID NO: 22-IGH CDR3
CAGRSSTAYYYIMDIW
23. SEQ ID NO: 23-IGH CDR3
CAGRSSTAYYYTMDIW
24. SEQ ID NO: 24-IGH CDR3
CAGVSYYYDSSGYYYEPFDYW
25. SEQ ID NO: 25-IGH CDR3
CAHGKLAGPFDSW
26. SEQ ID NO: 26-IGH CDR3
CAHGRYLDGAIDYW
27. SEQ ID NO: 27-IGH CDR3
CAHKKLFGELPDYW
28. SEQ ID NO: 28-IGH CDR3
CAHLTITFGGTPRDDAFDSW
29. SEQ ID NO: 29-IGH CDR3
CAHRLGPLANRAAYFDYW
30. SEQ ID NO: 30-IGH CDR3
CAHRQGYSYGIADYW
31. SEQ ID NO: 31-IGH CDR3-MBP Amino acid alignment from Adaptive Match
FLPRHRDTGILDSIGRFF
32. SEQ ID NO: 32-IGH CDR3-MBP Amino acid alignment from Adaptive Match
GFKGVDAQGTLSKIFKLG
33. SEQ ID NO: 33-IGH CDR3-MBP Amino acid alignment from Adaptive Match
PVVHFFKNIVTPRTPPP
34. SEQ ID NO: 34-IGH CDR3-MBP Amino acid alignment from Adaptive Match
PVVHFFKNIVTP
35. SEQ ID NO: 35-IGH CDR3-MBP Amino acid alignment from Adaptive Match
LPRHRDTGILDSIGRF
36. SEQ ID NO: 36-IGH CDR3-MBP Amino acid alignment from Adaptive Match
GFKGVDAQGTLSKIF
37. SEQ ID NO: 37-IGH CDR3-MBP Amino acid alignment from Adaptive Match
FKGVDAQGTLSKIFKLG
38. SEQ ID NO: 38-IGH CDR3-MBP Amino acid alignment from Adaptive Match
GVDAQGTLSKIFKLG
39. SEQ ID NO: 39-IGH CDR3-MBP Amino acid alignment from Adaptive Match
FKGVDAQGTLSKIFKLG
40. SEQ ID NO: 40-IGH CDR3-MBP Amino acid alignment from Adaptive Match
YKSAHKGFKGVDAQGTLSKIFKLG
41. SEQ ID NO: 41-IGH CDR3-MBP Amino acid alignment from Adaptive Match
GFKGVDAQGTLSKIF
42. SEQ ID NO: 42-IGH CDR3-MBP Amino acid alignment from Adaptive Match
KGVDAQGTLSKIFKLG
43. SEQ ID NO: 43-IGH CDR3-MBP Amino acid alignment from Adaptive Match
TGILDSIGRFFGGDRGAPKRG
44. SEQ ID NO: 44-IGH CDR3-MBP Amino acid alignment from Adaptive Match
FKGVDAQGTLSKI
45. SEQ ID NO: 45-IGH CDR3-MBP Amino acid alignment from Adaptive Match
VDAQGTLSKIFKLG
46. SEQ ID NO: 46-IGH CDR3-MBP Amino acid alignment from Adaptive Match
GILDSIGRFFGGDRGAPKRG
47. SEQ ID NO: 47-IGH CDR3-MBP Amino acid alignment from Adaptive Match
ILDSIGRFFGGDRGAPKR
48. SEQ ID NO: 48-IGH CDR3-MBP Amino acid alignment from Adaptive Match
GVDAQGTLSKIFKLG
49. SEQ ID NO: 49-Proteolipid Protein Antigen
LYGA
50. SEQ ID NO: 50-WURR-Combo CS for MBP isoform 5 range 118-131
GAEGQRPGFGYGGR
51. SEQ ID NO: 51-IGH CDR3
CARVLDWRAGSPTSPW
52. SEQ ID NO: 52-IGH CDR3
CARRMTVVAEYNFWSSYSSGPSWFDPW
53. SEQ ID NO: 53-IGH CDR3 Compliment Sequence
QDENPVVHFFKNIVTPRTPPPSQGKGR
54. SEQ ID NO: 54- IGH CDR3
CAKTRPHLVLVTVPVW
55. SEQ ID NO: 55-IGH CDR3 Compliment Sequence
TQDENPVVHFFKNIVT
56. SEQ ID NO: 56-Alpha/Beta Gliadin MM1 antigen peptide
TFLILALLAIVATTARIA VRVPVPQLQ
57. SEQ ID NO: 57-Alpha/Beta Gliadin MM1 antigen peptide
IPCRDVVLQQHSIAYGS
58. SEQ ID NO: 58-Alpha/Beta Gliadin MM1 antigen peptide
IPEQSRCQAIHNVVHAII
59. SEQ ID NO: 59-Prolamin antigen peptide
QILQQILQQQLIPCRDVVLQQPNIAHASS
60. SEQ ID NO: 60-Prolamin antigen peptide
IHNVIHAIILHHQ
61. SEQ ID NO: 61-Prolamin antigen peptide
NPQAQGFVQPQQLPQFEEIRNLALQTLPAMCNVYIPPYCSTTIAPFGIFS
62. SEQ ID NO: 62-Gamma Gliadin antigen peptide
TLLILTILAMAITIGTANIQVD
63. SEQ ID NO: 63-Gamma Gliadin antigen peptide
QGIDIFLPLSQHEQVGQGSLV
64. SEQ ID NO: 64-Gamma Gliadin antigen peptide
GIIQPQQPAQLEAIRSLVLQTLPSMCNVYVPPECSIMRAPFASIVAGIGGQ
65. SEQ ID NO: 65-Gamma Gliadin antigen peptide
HRDGVRRPQKRPSCIGCKGTH
66. SEQ ID NO: 66-Gamma Gliadin antigen peptide
YNLRR
67. SEQ ID NO: 67-Gamma Gliadin antigen peptide
DGVDLPPWFPPMVEGAAAEGDDG
68. SEQ ID NO: 68-Gamma Gliadin antigen peptide
GGDNHGRGRGRGRGRGGG
69. SEQ ID NO: 69-Gamma Gliadin antigen peptide
EGTWVAGVF
70. SEQ ID NO: 70-Gamma Gliadin antigen peptide
YNLRRGTALAIPQCRLTPLSRLPF
71. SEQ ID NO: 71-Gamma Gliadin antigen peptide
GPLRESIVCYF
72. SEQ ID NO: 72-Gamma Gliadin antigen peptide
VFLQTH
73. SEQ ID NO: 73-Gamma Gliadin antigen peptide
KDAIKDLVMTKPAPTCNIRV
74. SEQ ID NO: 74-Gamma Gliadin antigen peptide
FPPMVEGAAAEGDDG

What is claimed is:

1. A method of treating or preventing an autoimmune disease in a subject, the method comprising:
a. collecting a sample from the subject;
b. identifying one or more immunoglobulin heavy chain (IGH) complementarity determining regions (CDR) 3 within the sample;
c. determining a complementarity score (CS) between the IGH CDR3 and an epitope of the autoimmune disease, wherein the CS is based on electrostatic and hydrophobic interactions between the IGH CDR3 and the epitope; and
d. administering a therapeutic agent to the subject when the CS score is increased relative to a control subject.

2. The method of claim 1, wherein the autoimmune disease comprises Multiple Sclerosis (MS) or celiac disease.

3. The method of claim 1, wherein the subject is administered the therapeutic agent when the CS score is 6.0 or more.

4. The method of claim 1, wherein the epitope comprises a part of a whole antigen peptide.

5. The method of claim 1, wherein the therapeutic agent comprises an immunotherapeutic agent, a muscle relaxant agent, an analgesic, a plasma composition, a cell-based composition, or a combination thereof.

6. The method of claim 1, wherein the sample is a blood sample.

7. A method of diagnosing a subject with an autoimmune disease in a subject, the method comprising:
a. collecting a sample from the subject;
b. identifying one or more immunoglobulin heavy chain (IGH) complementarity determining regions (CDR) 3 within the sample;
c. determining a complementarity score (CS) between the IGH CDR3 and an epitope of the autoimmune disease, wherein the CS is based on electrostatic and hydrophobic interactions between the IGH CDR3 and the epitope; and
d. diagnosing the subject with the autoimmune disease when the CS score is increased relative to a control subject.

8. The method of claim 8, wherein the autoimmune disease comprises Multiple Sclerosis (MS) or celiac disease.

9. The method of claim 8, wherein the epitope comprises a part of a whole antigen peptide.

10. The method of claim 8, wherein the subject is administered a therapeutic agent when the CS score is 6.0 or more.

11. The method of claim 12, wherein the therapeutic agent comprises an immunotherapeutic agent, a muscle relaxant agent, an analgesic, a plasma composition, a cell-based composition, or a combination thereof.

12. The method of claim 8, wherein the sample is a blood sample.

13. A computer-implemented method comprising:
obtaining or determining, by at least one processor, an immune repertoire for a subject's blood sample;
programmatically identifying, by the at least one processor, one or more candidate epitopes corresponding with at least one known or unknown autoimmune disease, by using at least one chemical complementarity algorithm to determine a ratio or value indicating a number of times each of the one or more candidate epitopes complements one of a plurality of amino acids; and
determining, by the at least one processor, a disease state or condition of the subject and/or isolating at least one target epitope based, at least in part, on a frequency count and/or degree of correspondence between each respective candidate epitope and respective amino acid.

14. The computer-implemented method of claim 15, wherein the computer-implemented method comprises isolating at least one target epitope and further:
determining a statistical significance of the at least one target epitope based, at least in part, on a difference in weighted unique residue ratio (WURR) values outside the at least one target epitope relative to one or more control samples.

15. The computer-implemented method of claim 15, wherein identifying the one or more candidate epitopes comprises applying a sliding window analysis with respect to the one or more candidate epitopes and the plurality of amino acids.

16. The computer-implemented method of claim 15, further comprising, generating user interface data (e.g., graphical information, a report) based on the determined disease state or condition of the subject and/or isolated target epitope.

17. A system comprising:
at least one processor; and
a memory operably coupled to the at least one processor, wherein the memory has computer executable instructions stored thereon that, when executed by the at least one processor, cause the at least one processor to:
obtain or determine an immune repertoire for a subject's blood sample;
programmatically identify one or more candidate epitopes corresponding with at least one known or unknown autoimmune disease, by using at least one chemical complementarity algorithm to determine a ratio or value indicating a number of times each of the one or more candidate epitopes complements one of a plurality of amino acids; and
determine a disease state or condition of the subject and/or isolating at least one target epitope based, at least in part, on a frequency count and/or degree of correspondence between each respective candidate epitope and respective amino acid.
